(19) United States
(12) Patent Application Publication
(10) Pub. No.: US 2024/0390445 A1
(43) Pub. Date: Nov. 28, 2024

(54) METHOD OF TREATING CANCER

(71) Applicant: Native Ingredients and Health Research Organisation Pty Ltd, Tuggerah, New South Wales (AU)

(72) Inventors: Yuen Yee CHENG, Riverwood (AU); Quan Van VUONG, Ourimbah (AU); Karl Lijun Qin, Killara (AU)

(73) Assignee: Native Ingredients and Health Research Organisation Pty Ltd, Tuggerah, New South Wales (AU)

(21) Appl. No.: 18/696,920

(22) PCT Filed: Sep. 30, 2022
(86) PCT No.: PCT/AU2022/051174

§ 371 Date: Mar. 28, 2024

(30) Foreign Application Priority Data
Oct. 1, 2021 (AU) 2021903147

(51) Int. Cl. A61K 36/61; A23L 21/25; A61K 9/00; A61K 9/14; A61K 35/644; A61K 45/06; A61P 35/00

(52) U.S. Cl. CPC A61K 36/61; A23L 21/25; A61K 9/0053; A61K 9/148; A61K 35/644; A61K 45/06; A61P 35/00

(57) ABSTRACT

The present disclosure relates generally to extracts from a plant of the genus Leptospermum, compositions comprising the extracts, and methods of producing the extracts. The present disclosure also relates to methods and uses of these extracts in the treatment or prevention of cancer, metabolic syndromes, diseases or conditions associated with oxidative stress, and maintaining healthy glucose levels in a subject in need thereof.

### FIELD OF THE INVENTION

The present disclosure relates generally to extracts from a plant of the genus Leptospermum, compositions comprising the extracts, and methods of producing the extracts. The present disclosure also relates to methods and uses of these extracts in the treatment or prevention of cancer, metabolic syndromes, diseases or conditions associated with oxidative stress, and maintaining healthy glucose levels in a subject in need thereof.

### BACKGROUND

The genus Leptospermum J. R. et G. Forst. belongs to the Myrtaceae family, commonly referred to as the tea tree family. To date, 87 Leptospermum species have been identified and over 80 species are endemic to Australia with the greatest diversity in the south of the continent, and a few species are native to New Zealand and Southeast Asia. Leptospermum plants have been used for the production of Leptospermum honey, also known as Manuka honey.

The Leptospermum honey is unique as compared to standard honey because it contains a compound called methylglyoxal (MGO), which is thought to underlie its antimicrobial properties. More recently, Leptospermum honey has been linked with various health benefits, such as treatment of burns, cataracts, ulcers and wound healing.

Numerous Leptospermum species have been used as traditional herbal medicines for the treatment of various ailments, such as skin diseases, urinary tract conditions, abdominal discomfort, cough, colds and gum diseases, indicating that this species may potentially contain a number of unidentified bioactive compounds having therapeutic activity. Certain bioactive compounds, such as the previously mentioned MGO, and additionally phenolic compounds and terpenes have been found in Leptospermum. However, the link between the presence of these compounds and the therapeutic utility of Leptospermum species is not well understood. One factor that may contribute to this lack of understanding is that these bioactive compounds are highly susceptible to degradation due to exposure to enzymes, oxidation, light, heat, and oxygen. Additionally, these bioactive compounds can be degraded as a result of improper extraction steps or during subsequent storage in inappropriate environmental conditions.

Various methods of extracting bioactive compounds from Leptospermum are known in the art. However, these methods do not properly take into account factors that affect the stability, and thus the physiological activity of these bioactive compounds. For example, a number of extraction methods utilise heat, however a number of bioactive compounds are sensitive to elevated temperatures and application of heat can either reduce their physiological activity or degrade these bioactive compounds. Additionally, the art is focused primarily on Manuka honey produced by bees when pollenating Leptospermum plant species, as distinct from any bioactives that might be present in Leptospermum plant material itself.

It would be desirable to identify new novel processes for extracting and preparing useful substances from plants of the genus Leptospermum comprising desirable extraction conditions allowing for effective extraction of bioactive compounds having potential physiological and biochemical activity. Further, it would be desirable to identify new uses for Leptospermum extracts for improving human health.

Any publications mentioned in this specification are herein incorporated by reference. However, if any publication is referred to herein, such reference does not constitute an admission that the publication forms a part of the common general knowledge in the art in Australia or any other country.

### SUMMARY OF THE INVENTION

The inventors have identified that extracts prepared from Leptospermum exhibit novel biological and therapeutic activity, useful for the treatment of disease and conditions as well as for improving human health.

Accordingly, an aspect of the invention provides a method of treating cancer in a subject in need thereof comprising administering a composition comprising a polar solvent extract from a plant of the genus Leptospermum. In a further, related aspect there is also provided the use of a polar solvent extract from a plant of the genus Leptospermum in the manufacture of a medicament for treating cancer in a subject in need thereof.

Preferably, the polar solvent extract is an aqueous extract.

A further aspect of the invention provides a method for treating mesothelioma in a subject in need thereof comprising administering a composition comprising a polar solvent extract from a plant of the genus Leptospermum. In a further, related aspect there is also provided the use of a polar solvent extract from a plant of the genus Leptospermum in the manufacture of a medicament for treating mesothelioma in a subject in need thereof.

The term “bioactive compounds” refers to active components that are able to exert effects on innate biological pathways in a subject, for example by activation or repression of physiological pathways.

A further aspect of the invention provides a method of treating a metabolic syndrome in a subject in need thereof comprising administering an extract from a plant of the genus Leptospermum. In a further, related aspect there is also provided the use of an extract from a plant from genus Leptospermum in the manufacture of a medicament for treating a metabolic syndrome in a subject in need thereof.

In embodiments, the metabolic syndrome is selected from hyperglycemia, dyslipidemia, obesity and/or adiposity.

Yet another aspect of the invention provides a method of maintaining healthy glucose levels in a subject in need thereof comprising administering an extract from a plant of the genus Leptospermum. In a further, related aspect there is also provided the use of an extract from a plant of the genus Leptospermum in the manufacture of a medicament for maintaining healthy glucose levels in a subject in need thereof.

A further aspect of the invention provides a method of treating or preventing a disease or condition associated with oxidative stress in a subject comprising administering an extract from a plant of the genus Leptospermum to a subject in need thereof. In a further, related aspect there is also provided the use of an extract from a plant of the genus Leptospermum in the manufacture of a medicament for treating or preventing a disease or condition associated with oxidative stress.

In some embodiments, the extract comprises phytochemicals. In some embodiments, the phytochemicals, or actives, are phenolic compounds.

The inventors have found that the total phenolic component (TPC) of the extract comprises phytochemicals, including phenolic compounds in particular. The phytochemicals (actives) have been shown to have potent antioxidative properties.

In some embodiments, the extract comprises any one or more actives selected from the group consisting of: aromadendrin glucoside, kaempferol rhamnoside, quercetin rhamnoside, vindoline, 5-dihydroxy-6-methyl-7-methoxyflavanone, and methylglyoxal. In some embodiments, the extract comprises at least 3, at least 4, at least 5 or all 6 of these actives.

In some embodiments, the extract is in powder form.

In some embodiments, the extract is in encapsulated form.

The term “encapsulation” refers to the process of coating compounds or molecules. Encapsulation has been shown to improve bioaccessibility and bioavailability of certain compounds, for example, bioactive compounds that are sensitive to digestion in the gastrointestinal tract.

While the present disclosure describes the preparation and use of extracts from Leptospermum, specific actives may be isolated from the Leptospermum extract (and optionally purified), and those actives may also be used in the methods of the present disclosure. In this context, the term “extract” also extends to an isolated active compound from the extract.

The inventors have also identified an improved method for producing an extract from a plant of the genus Leptospermum, which utilises a polar solvent to extract bioactive compounds (also referred to herein as actives) that would typically be degraded in conventional extraction processes known in the art.

Accordingly, an aspect of the invention provides a method of producing an extract, the method comprising:
contacting fragmented plant material from a plant of the genus Leptospermum with a polar solvent for a period of at least about 10 minutes to about 2 hours and at a temperature of at least 50° C. to 120° C.;
separating remaining solids from the polar solvent to yield the extract.

The extract is produced with the use of a polar solvent, such as water, ethanol, methanol, acetone, or mixtures thereof. The fragmented plant material from a plant of the genus Leptospermum is contacted with a polar solvent in order to extract the polar solvent-soluble chemical compounds or actives from the plant material into the polar solvent. The extract thus obtained can be further treated to either remove or reduce the volume of polar solvent to produce a concentrated extract. The extract may also be subjected to further steps, for example purified to obtain individual or groups of bioactive compounds, dried to obtain powdered extract, or encapsulated to get the encapsulated extract.

The polar solvent may be selected from the group consisting of water and polar organic solvents.

In a preferred embodiment, the polar solvent comprises water. In most preferred embodiments, the polar solvent is water.

Examples of suitable polar organic solvents include short-chain polar organic solvents containing between 1 and 6 carbon atoms, such as short chain alcohols (C1-C6 alcohols, examples being methanol or ethanol), glycerine and acetone. The polar solvents are preferably non-toxic. Combinations of solvents may also be used, such as water and alcohol to yield a hydroalcoholic extract, or water and glycerine to yield a hydroglycolic extract. In another alternative sequential extraction using different solvents may be practiced, and the extracts combined.

The extracts so produced are distinct from essential oils produced from the Leptospermum plant species. Essential oils are extracts of the volatile components of the plant material, those volatile components having been extracted by distillation or similar processes that involve collection of volatile components of the plant. The volatile components are those capable of separation from the plant material through distillation on the application of heat. The essential oil components may comprise non-polar compounds. Such compounds may include aromatic compounds, terpenes and other volatile hydrocarbon products. The term “volatile organic compound” refers to an organic compound that has a boiling point (an initial boiling point) in the range of 50 to 250° C., measured at atmospheric pressure. The nature of the plant components that are extracted into the polar-solvent extracts (e.g. aqueous extracts) are non-volatile compounds, which are different to volatile compounds in essential oils.

The balance of the composition is made up of other plant products and byproducts, and optionally the solvent (e.g. water).

The term “other plant products and byproducts” refers to naturally occurring byproducts and other substances arising during the extraction process, and may include hydrocarbon compounds, amino acids, chlorophyll, or any other bioactive compounds or actives. Examples of other plant products and byproducts present in the extract include, but are not limited to, alkaloids, glycosides, steroids, triterpenoids, tannins, flavonoids and proteins. These plant products and byproducts are typically polar solvent-soluble components. Plant solids (i.e. the fibrous plant material or pulp) are not within the scope of this term.

In a preferred embodiment, the fragmented plant material is dried to a moisture content not greater than 10% prior to contacting with the polar solvent.

Combining this step with the other steps of the process, according to this preferred embodiment, the method of producing an extract comprises:
drying fragmented plant material from a plant of the genus Leptospermum to a moisture content not greater than 10%;
contacting the fragmented dried plant material with a polar solvent for a period of at least about 10 minutes to about 2 hours and at a temperature of at least 50° C. to 120° C.; and
separating remaining solids from the polar solvent to yield the extract.

In a further aspect, there is provided a composition comprising a polar solvent extract from a plant of the genus Leptospermum. Expressed in a different manner, provided in the present aspect is a composition comprising bioactive compounds from a plant of the genus Leptospermum. The composition comprises polar solvent-soluble bioactive compounds from the plant of the genus Leptospermum, or aqueous-soluble bioactive compounds from the plant of the genus Leptospermum, specifically.

In some embodiments, the composition comprises any one or more actives selected from the group consisting of: aromadendrin glucoside, kaempferol rhamnoside, quercetin rhamnoside, vindoline, 5-dihydroxy-6-methyl-7-methoxyflavanone, and methylglyoxal (MGO). In some embodiments, the composition comprises at least 3, at least 4, at least 5 or all 6 of these actives.

The balance of the composition is made up of other plant products and byproducts, and optionally the solvent.

The term “other plant products and byproducts” is as defined above.

In some embodiments, the polar solvent extract is an aqueous extract.

In some embodiments, the composition comprises a polar solvent extract of plant material of the genus Leptospermum. In some embodiments, the composition comprises an aqueous extract of plant material of the genus Leptospermum. In some embodiments, the composition comprises a polar solvent extract of dried plant material of the genus Leptospermum. In one specific embodiment, the composition comprises an aqueous extract of dried plant material of the genus Leptospermum In some embodiments, the polar solvent is removed following extraction.

In some embodiments, the composition further comprises a honey component. Preferably, the honey component is a dried honey product or a honey powder.

### BRIEF DESCRIPTION OF FIGURES

Embodiments of the disclosure will now be described by way of example only with reference to the following non-limiting Figures. Extracts according to the present disclosure are referred to as ‘Leptospermum extract’ or ‘LEP’ or ‘Honey’ in the Figures and the description of the Figures that follows.

FIG. 1 shows the effects of Leptospermum extract on rates of cancer cell proliferation as compared to Manuka honey. The figure comprises line graphs showing the results of a cytotoxic assay on various cancer cell lines, and compares the effects of UMF 10+Manuka honey (UMF 10+), Honey formula comprising 2.5% Leptospermum extract (Honey), and pure Leptospermum extract (LEP). The line graphs illustrate the cytotoxic activity on the cancer cell lines (as measured by the percentage of cells remaining at the end of the assay) versus different concentrations. The results indicate that LEP was cytotoxic to cancer cells at most concentrations, killing nearly all the cancer cells by the end of the assay. In contrast, UMF 10+ and Honey were only cytotoxic at much higher concentrations.

FIG. 2 shows the effects of Leptospermum extract on tumor cell growth. The figure comprises bioluminescence photographs of a rat animal model of mesothelioma (MSTO) and lung cancer, where a lighter hue indicates a high density of tumor cells (i.e. a larger size of tumor) and the presence of an overlay on the photograph indicates a presence of tumor cells in the rat. The top panel of photographs illustrates that rats that were fed with daily LEP supplement (MSTO+LEP) had a significantly reduced tumor size and density by day 19 as compared to the control (MSTO), which persevered until day 30. The bottom photographs illustrate that the presence of lung cancer cells was significantly reduced in the LEP treated group (labelled ‘Treatment’) as compared to the controls.

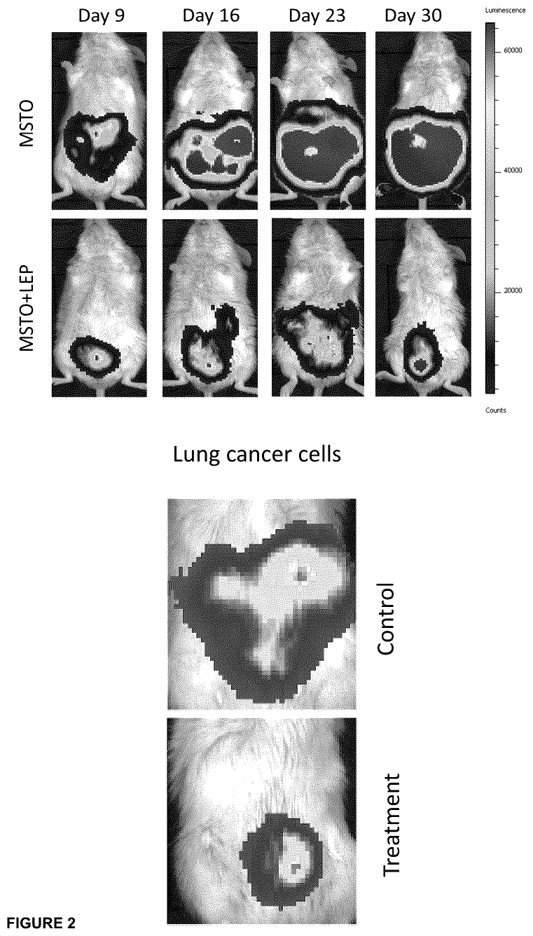

FIG. 3 shows the analysis of the bioluminescence photographs of FIG. 2 as well as a H1975 control group. The figure comprises column graphs showing a representative tumor cell count at day 9, day 16, day 23 and day 30 for both MSTO and H1975 control groups and MSTO+LEP, and H1975+LEP treatment groups.

FIG. 4 shows the effects of LEP treatment on tumor size and weight. The figures on the top are photographs showing tumours removed from rats from both the MSTO control group and MSTO+LEP treatment group once the rats had succumbed to the effects of the cancer (30 days in the MSTO controls vs 33 days in the MSTO+LEP treatment group). The column graph on the bottom illustrates that the tumor weight to body weight percentage was decreased in LEP treated rats as compared to the untreated controls.

FIG. 5 shows the effects of LEP treatment on animal body weight as an index of general health. The figure comprises a line graph showing the changes in body weight remaining over time (100% representing no change in body weight from day 0). The MSTO+LEP treatment group exhibited significantly increased body weight at days 13, 15 and 19 compared to untreated rats, suggesting that the LEP treated rats exhibited better general health. * indicates a p-value 50.05, ** indicates a p-value 50.01.

FIG. 6 shows the effects of LEP supplementation on animal body weight. The figure comprises a line graph and column graph showing the changes in body weight in C57 mice that were fed a normal diet as controls as compared to C57 mice that were fed honey formula with 20 mg/mouse/day Leptospermum extract (labelled “Honey” in the figure). Mice fed with the honey formula and leptospermum extract exhibited a decrease in body weight at day 7 that persevered until day 28 as compared to the control mice.

FIG. 7 shows the effects of LEP supplementation on animal body fat mass. The figures comprises photographs of DEXA scans graph showing the changes in body weight after 28 days of feeding in C57 mice that were fed a normal diet as controls as compared to C57 mice that were fed honey formula with 20 mg/mouse/day Leptospermum extract (“Honey”). Mice fed with the honey formula and Leptospermum extract exhibited a reduced body fat mass (16% in Honey vs 19% in control).

FIG. 8 shows the analysis of the DEXA scan photographs of FIG. 7. Mice that were fed with honey supplemented with LEP exhibited a slight increase in lean mass and a corresponding decrease in fat mass as compared to control mice fed with a normal diet.

FIG. 9 shows the liver toxicity analysis after 28 days of feeding in C57 mice that were fed a normal diet as controls as compared to C57 mice that were fed honey formula with 20 mg/mouse/day leptospermum extract (“Honey”). Both groups exhibited low levels of the liver enzymes aspartate transaminase (AST) and alanine transaminase (ALT), suggesting that the Leptospermum extract did not cause liver damage.

FIG. 10 shows liver histology analysis after 28 days of feeding in C57 mice that were fed a normal diet as controls as compared to C57 mice that were fed honey formula with 20 mg/mouse/day Leptospermum extract (Honey). The figure comprises photomicrographs of hematoxylin and eosin stained liver samples. Histological analysis revealed a normal liver appearance in both groups.

FIG. 11 shows the fasting blood glucose concentration after 28 days of feeding in C57 mice that were fed a normal diet as controls as compared to C57 mice that were fed honey formula with 20 mg/mouse/day Leptospermum extract (“Honey”). The figure comprises a column graph illustrating that mice fed with the honey formula and Leptospermum extract after a 6 hour fasting period exhibited decreased blood glucose levels as compared to control animals.

FIG. 12 shows a HPLC chromatogram of the Leptospermum extract, with the relevant peaks correlated to chemical constituents or actives contained within the Leptospermum extract. Two peaks are identified on the chromatogram as quercetin and kaempferol, as shown by the black arrows.

FIG. 13 shows the effects of the Leptospermum extract on migration on a cancer cell line, as measured using a scratch (wound-healing) assay. The Figure comprises microscope images of the V40 cancer cell line, and shows that V40 cells treated with Leptospermum extract had a significant wound gap at the 24 hour time-point when compared to the untreated control, suggesting Leptospermum extracts not only inhibit cancer cell proliferation but also migration. The wound gap is illustrated in the squares.

FIG. 14 shows the effects of the Leptospermum extract on a cancer cell line after 24 hours of treatment. The figure comprises microscope images of the V40 cancer cell line before (top left image) and after (bottom left image) treatment with the Leptospermum extract. The microscope images (circled) show that cancer cells undergo cell death after treatment. Magnified microscope images show a close-up of a cancer cell before (top right image) and after (bottom right image) treatment with the Leptospermum extract.

### DETAILED DESCRIPTION

The following is a detailed description of the disclosure provided to aid those skilled in the art in practicing the present disclosure. The person skilled in the art may make modifications and variations in the embodiments described herein without departing from the spirit or scope of the present disclosure.

The present invention relates to the methods of treating or preventing cancer, a metabolic syndrome, diseases or conditions associated with oxidative stress, or maintaining healthy glucose levels in a subject, comprising administering a composition comprising a polar solvent extract from a plant of the genus Leptospermum in the treatment.

The term “polar solvent extract” refers to an extract that is obtained by a process of extraction from plant material with the use of a polar solvent. The extract may be in the form of a concentrated extract, a refined extract, or a purified extract. It will be understood that the term “extract” is accordingly not limited to the original extraction product, but extends to such downstream products. Preferably, the polar solvent extract is an aqueous extract.

The term “aqueous extract” has the same definition as “polar extract”, but with the polar solvent limited to a water-containing solvent. An aqueous extract comprises water-soluble chemical compounds or bioactive compounds (or “actives”) that are removed from, in this case, plant material or fragments of a plant of the genus Leptospermum.

Throughout the detailed description, reference is made to “aqueous extracts” since it is a preferred embodiment. However, it should be understood that the features described with reference to “aqueous extracts” extend equally to other polar solvent extracts. The description should not be read as suggesting that the invention is limited to aqueous extracts only.

The present invention also relates to the use of the aqueous extracts in the manufacture of a medicament for use in the prevention or treatment or prevention of cancer, mesothelioma, a metabolic syndrome, a disease or condition associated with oxidative stress, or maintaining healthy glucose levels in a subject.

In embodiments, the metabolic syndrome is selected from hyperglycemia, dyslipidemia, obesity and/or adiposity.

The term “mesothelioma” refers to an uncontrolled cell growth that occurs in the mesothelium. The mesothelium is a thin layer of tissue that covers the majority of the body's internal organs.

In embodiments, the mesothelioma is a mesothelioma of the lung, abdomen or heart.

The term “maintaining healthy glucose levels” refers to maintaining a subject's glucose levels within a healthy range. The determination of a healthy range of glucose levels may differ from patient to patient as well as the attending physician, and could be affected by the exact clinical and physiological circumstances, and even the timing of the glucose level measurement and fasting status. However, a healthy blood glucose level would typically be between about 4.0-7.8 mmol/L.

The term “hyperglycemia” is also known as high blood glucose, and refers to when the subject's glucose levels are in excess to their healthy glucose levels.

The person skilled in the art will readily understand how to assess and quantify the diseases or conditions discussed herein or a characteristic feature thereof, and be able to do so without difficulty or undue burden, for example using methods set out in the present examples. For instance, the diagnosis of lung cancer includes, but is not limited to: (i) medical imaging, for example x-rays or CT scans of the lungs in order to determine tumorous growth or abnormal lesions; (ii) sputum cytology to reveal the presence of lung cancer cells, or (iii) biopsy tissue sample, including the use of bronchoscopy or needle biopsy to collect cells from the lungs and surrounding lymph nodes to diagnose potentially cancerous growth.

The inventors have identified that extracts having bioactive compounds or actives from Leptospermum have therapeutic utility in these diseases. The use of an aqueous extract is advantageous because it can be administered directly to a subject in a safe manner. In contrast, when a chemical-based extraction process is used, the extracts need to be purified prior to administration. While a number of purification methods are known in the prior art, there remains the risk that small or trace amounts of the chemicals remain which may cause long-term side effects or health issues.

The use of Leptospermum extracts in therapy have been reported previously, with particular regard to their antimicrobial and anti-inflammatory properties. However to date, there have been no studies showing the use of Leptospermum extracts in treating the diseases and conditions and improving human health as presently described herein. The inventors are also the first to show the advantageous effects of a Leptospermum aqueous extract as described herein.

In some embodiments, the plant of the genus Leptospermum is selected from the group consisting of, but not limited to, Leptospermum scoparium, Leptospermum liversidgei, Leptospermum polygalifolium, Leptospermum laevigatum, Leptospermum continentale, Leptospermum amboinense, Leptospermum arachnoides, Leptospermum brachyandrum, Leptospermum brevipes, Leptospermum coriaceum, Leptospermum deuense, Leptospermum epacridoideum, Leptospermum juniperinum, Leptospermum lanigerum, Leptospermum luehmannii, Leptospermum macrocarpum, Leptospermum minutifolium, Leptospermum multicaule, Leptospermum myrsinoides, Leptospermum nitidum, Leptospermum obovatum, Leptospermum oligandrum, Leptospermum parvifolium, Leptospermum petersonii, Leptospermum polygalifolium ‘Pacific Beauty’, Leptospermum purpurascens, Leptospermum rotundifolium, Leptospermum rotundifolium ‘Julie Ann’, Leptospermum sericeum, Leptospermum speciosum, Leptospermum spectabile, Leptospermum spinescens, Leptospermum squarrosum, Leptospermum trinervium, Leptospermum turbinatum, Leptospermum variabile, and Leptospermum wooroonooran.

In the Examples described herein, the plant of the genus Leptospermum is Leptospermum polygalifolium.

In some embodiments, the extract comprises any one or more actives selected from the group consisting of: aromadendrin glucoside, kaempferol rhamnoside, quercetin rhamnoside, vindoline, 5-dihydroxy-6-methyl-7-methoxyflavanone, and methylglyoxal.

While the inventors have isolated the aqueous extracts naturally from Leptospermum plants, the methods and uses of the invention also encompass the use of separately purified compositions comprising the actives described in embodiments of the invention. Without being bound by theory, the inventors theorise that the presence of the actives may also provide a form of synergistic relationship between the actives and/or excipients present in the aqueous extracts, which could contribute to the therapeutic and biological activity of these naturally-occurring compositions.

The term “subject” encompasses mammals and non-mammals. Examples of mammals include, but are not limited to, any member of the Mammalian class: humans; non-human primates such as chimpanzees, and other apes and monkey species; farm animals such as cattle, horses, sheep, goats, swine; domestic animals such as rabbits, dogs, and cats; and laboratory animals including rodents, such as rats, mice and guinea pigs, and the like. Examples of non-mammals include, but are not limited to, birds, fish and the like. Preferably, the subject is a human.

The terms “treat”, “treating” or “treatment” refer to both therapeutic and prophylactic or preventative measures, wherein the aim is to reduce the symptoms of, prevent or ameliorate the disease or condition or a characteristic feature thereof in a subject. Alternatively, the term may also be used to refer to the slowing down of, or lessening or postponing the progression of disease or a characteristic feature thereof in a subject. Subjects in need of treatment include those having the disease or condition or a characteristic feature thereof, or preventing those who have a propensity or risk of developing said disease or condition or a characteristic feature thereof. Treatment also encompasses ameliorating or preventing the worsening of existing symptoms of the disease or condition, the underlying metabolic or physiological processes leading to development of the disease or condition, and causing regression of the disease or condition.

The term “treating cancer” refers to, for example, reducing the number of cancerous cells, reducing tumour size, reducing the rate of invasion of the cancer cells into other organs from the originating site, reducing the rate of tumour metastasis or slowing down the tumour growth rate. The achievement of treatment can be measured by a number of ways known in the art, for example progression free survival, disease free survival, or overall survival. The term “progression free survival” refers to a period of time during and after treatment in which the cancer does not grow i.e. progress, or the amount of time the patient experiences a complete or partial response, or put alternatively, the time during which the disease progression is stable. The term “disease free survival” refers to a period of time during or after treatment by which the patient is free from the disease. The term “overall survival” refers to a relative period of extended lifespan as compared to untreated or control patients.

The term “preventing cancer” refers to, for example, delaying or halting the onset of cancer in a subject, inhibiting of cancer spread (i.e. metastasis) or inhibiting tumour growth.

The term “tumour” refers to a neoplasm or tissue mass that is malignant or potentially malignant, including primary tumours and secondary neoplasms. Solid tumours are tumour masses that do not contain liquid or cysts. Examples of tumours are sarcomas, carcinomas, and lymphomas.

The term “tumour size” refers to the total size of a tumour measured, for example using length and width dimensions, or by tumour cell density counts. Tumour sizes can be measured by a number of ways known in the art, for example by callipers once removed from the subject or using imaging techniques such as ultrasound, CT or MRI scans.

The term “effective amount” or “therapeutically effective amount” refers to a sufficient amount of agent to provide the desired therapeutic or biological effect. The effect includes, but is not limited to, the reduction and/or alleviation of the causes, symptoms or changes in underlying physiological pathways that underlie the development of a disease or condition.

The use of the term “effective amount” in some embodiments refers to a sufficient amount of active compound that provides a clinically relevant change in disease status, presence or absence of symptoms, or medical condition. The person skilled in the art will typically be able to assess and quantify the required “effective amount” of an active compound required on an individual, case-by-case basis using routine knowledge in the art.

The term “medicament” refers to a form of formulation or preparation, including medical preparations, that are suitable for administration to a subject. The medicament may comprise other active drugs or therapeutic agents, and one or more pharmaceutical excipients. Alternatively, the medicament may also refer to a food composition or nutraceutical composition comprising the extract that is intended to be delivered to a subject.

The actual dosage employed may vary depending on the exact requirements of the patient, and severity of the disease or condition being treated. The appropriate dosage regimen can be assessed by a person skilled in the art, for example the total daily dosage required may be divided into multiple administrations throughout the course of the day.

The administration method or route for administering the aqueous extracts from a plant of the genus Leptospermum will typically be able to be assessed at the discretion of the health professional or medical practitioner. Factors that can affect the exact administration method include the subject's disease status, age, general health, convenience, contraindications with other pharmaceutical agents or drugs being taken by the subject, and other physiological factors. In some embodiments, the aqueous extracts are administered subcutaneously, intravenously, orally, and parenterally. Preferably, the aqueous extracts are administered orally.

The aqueous extracts from a plant of the genus Leptospermum may be administered to a patient in substantially natural form, since the extracts may be produced using naturally occurring and safe solvents. The aqueous extracts may also undergo one or more further purification and/or filtration steps. These may be useful in order to remove by-products of the extraction process if necessary. Additionally, the aqueous extracts may also be formulated into a composition suitable for administration to a subject. Preferably, the composition is sterile. In embodiments, the composition is pyrogen-free.

In embodiments, the extract is a dried aqueous extract. The extract may be in powder form. The dried aqueous extract, or powdered extract, may be combined with a suitable carrier for oral administration to a subject. The oral carrier may be selected from any of the known oral carriers in the art, examples of which include saccharides, carbohydrates (including carbohydrate polymers), edible proteins, semi-solid gels, pastes, ointments, jellies, waxes, oils, lipids, and oil-in-water or water-in-oil emulsions. The powdered extract may be compressed into tablet form with a suitable carrier, incorporated into capsules, encapsulated, packaged in a powder sachet, or otherwise. Such carriers can be selected from pharmaceutically acceptable excipients and auxiliaries. The term “pharmaceutically acceptable carrier” or “pharmaceutically acceptable vehicle” encompasses any of the standard pharmaceutical carriers, solvents, surfactants, or vehicles. Suitable pharmaceutically acceptable vehicles include aqueous vehicles and nonaqueous vehicles. Standard pharmaceutical carriers and their formulations are described in Remington's Pharmaceutical Sciences, Mack Publishing Co., Easton, PA, 19th ed. 1995.

In embodiments, the extract is in encapsulated form.

Various materials for and methods for encapsulating the extract are known in the art. Some examples include carbohydrate polymers (starch, gums, alginate, carrageenan), and proteins (gluten, protein isolate, casein, whey protein).

In this disclosure, in particular as detailed in Example 4, the therapeutic utility of aqueous extracts from a plant of the genus Leptospermum when delivered to a mouse model of cancer using severe combined immunodeficiency (SCID) mice were investigated. This mouse model is well accepted in the art as a pre-clinical model in cancer biology. SCID mice are immunocompromised animals and exhibit a propensity to develop disease, and are thus valuable models in cancer studies. Here, the inventors utilised intraperitoneal injections of human mesothelioma cancer and lung cancer cell lines in 10-week old SCID mice to trigger tumour growth.

Accordingly, in an embodiment, the cancer is mesothelioma.

Without being bound to theory, the inventors envisage that the anti-cancer effects of the Leptospermum extracts are related to reduced cancer cell migration and inducing cancer cell death, following treatment with the Leptospermum extracts.

In embodiments, the methods or uses according to other aspects of the invention further comprises the administration an additional therapeutic agent to the subject, or use of an additional therapeutic agent in the manufacture of a medicament, where the additional therapeutic is selected from the group consisting of: a chemotherapeutic agent, an anti-inflammatory agent, an immunomodulatory agent, a neurotropic factor, an agent for treating a cardiovascular disease, an agent for treating liver disease, an anti-viral agent, an agent for treating hyperglycemia, and an agent for treating immunodeficiency disorders.

In this disclosure, in particular as detailed in Example 5, the antioxidative properties of aqueous extracts from a plant of the genus Leptospermum were investigated. The aqueous extracts were found to have a high total phenolic content (TPC) and exhibited potent antioxidative properties in the ferric reducing antioxidant power (FRAP) in vitro assay. This supports the view that the aqueous extracts are useful in treating or preventing a disease or condition associated with oxidative stress in a subject in need thereof.

The balance between oxidants and antioxidants in human physiology is beneficial to prevent underlying damage to cells, tissues and organs. Cardiovascular diseases such as coronary heart disease, hypertension and stroke, and other diseases including inflammatory disorders (such as arthritis), neurodegenerative disorders (such as Parkinson's or Alzheimer's disease) and cancer are thought to be underpinned by reactive oxygen species leading to oxidative stress and chronic cellular injury. Increased amounts of reactive oxygen species including free radicals can also damage DNA. Antioxidants act by scavenging reactive oxygen species from the body, reducing the level of oxidative stress in the body. Some studies indicate that early use of antioxidants may also act prophylactically and prevent the disease from developing in the first place.

The Leptospermum extract is shown in the Examples to be one that results in cell death of at least 50% in cancer cell lines remaining following a cytotoxic assay (cell proliferation). The percentage of cancer cells that have undergone cell death in some embodiments is at least 70%, or even at least 80%.

The Leptospermum extract is shown to be one that provides a reduction in tumour cell count in cancer subjects of at least 10% as compared to controls. The reduction in tumour cell count in some embodiments is at least 20%, or even at least 50%.

The Leptospermum extract is shown to be one that provides a reduction in weight of tumours in cancer subjects of at least 1% relative to body weight, as compared to controls. The reduction in weight of tumours in some embodiments is at least 2%, or even at least 3% relative to body weight.

The Leptospermum extract is shown to be one that provides an increase in remaining body weight in cancer subjects of at least 1% as compared to controls. The increase in remaining body weight in some embodiments is at least 2%, or even at least 5%.

The Leptospermum extract is shown to be one that provides a reduction in body weight of at least 1% as compared to controls. The reduction in body weight in some embodiments is at least 2%, or even at least 5%.

The Leptospermum extract is shown to be one that provides a reduction in fat mass of at least 1% as compared to controls. The reduction in fat mass in some embodiments is at least 2%, or even at least 5%.

The Leptospermum extract is shown to be one that provides a reduction in glucose levels of at least 1 mmol/L as compared to controls. The reduction in glucose levels in some embodiments is at least 2 mmol/L, or even at least 3 mmol/L.

The Leptospermum extract is shown to be one that reduces cell migration of cancer cells in a wound-healing (scratch) assay by at least 10% compared to controls. The reduction in cell migration of cancer cells in some embodiments is at least 20%, or even at least 50%.

The Leptospermum extract is shown to be one that provides a basal respiratory capacity of at least 400 pmol/min as compared to controls, based on the Seahorse XF Cell Mito Stress Test. The basal respiratory capacity in some embodiments is at least 500 pmol/min, or even at least 600 pmol/min.

The Leptospermum extract is shown to be one that has a total phenolic content of at least 100 mg/gram of gallic acid (mg of gallic acid equivalent per g dry weight), as determined using the Folin-Ciocalteu method. The total phenolic content in some embodiments is at least 125 mg gallic acid/g, or even at least 150 mg gallic acid/g.

The Leptospermum extract is shown to be one that provides a ferric-reducing antioxidant potential (FRAP) of at least 500,000 mg/ml. The FRAP may, in some embodiments, be at least 700,000 mg/ml, or even at least 1,000,000 mg/ml.

In embodiments, the composition is a food composition. Preferably, the food composition comprises a honey product. Preferably, the honey product is from Leptospermum scoparum.

The term “food composition” refers to a composition that is suitable for ingestion (that is, consumable or drinkable). The food composition may be in liquid, semi-solid, or solid form, for example, in the form of a gel, syrup, paste, powder, snack, or drink.

The term “honey product” refers to honey in substantially pure liquid or semi-solid form (such as a gel, a traditional flowable liquid honey form, or in crystalline honey form), honey in combination with other common food additives, or honey derivatives such as dried honey or powdered honey, or combinations thereof.

Various methods for preparing dried honey are known in the art. Exemplary methods for producing dried honey include air drying, evaporation and cooling. Dried honey may also be produced in accordance with the method disclosed in New Zealand patent no. 556724.

In some embodiments, the dried honey has a moisture content of less than about 4% by weight compared to the total weight of the dried honey.

In some embodiments, the dried honey has a moisture content of about 1%, about 2% or about 3% w/w of the total weight of the dried honey.

In some embodiments, the honey is a manuka honey having an UMF score of at least 10. In embodiments, the honey is a manuka honey having an UMF of 10, 15, 18 or 20. Admixing or formulating the aqueous extracts into a food composition is advantageous for ease of administration to the subject. For example, patients in advanced stages of cancer may find consuming liquids difficult and mixing the extracts into a food composition comprising a honey product may be more desirable. In the alternative, formulating the aqueous extracts with food products such as honey may be more desirable for administration to subjects who are on a daily administration regime, for compliance and comfort reasons. In addition, formulating the aqueous extracts with food products can improve the properties and quality of the food products.

In embodiments, the Leptospermum extract is present in the food composition in an amount of about 0.01 to 20 wt %, based on the total weight of the food composition. To account for the impact that any remaining solvent (i.e. water or other polar solvent, if any remains) may have on the amount of the Leptospermum extract, the amount is calculated by reference to the solvent-free portion of the extract component that is present in the food composition. The amount of the Leptospermum extract may be a minimum of 0.01, 0.1, 1, 2, 3, 4 or 5% of the food composition. The amount of Leptospermum extract may in some embodiments be not more than 20%, 15%, 10%, 5%, 4%, 3%, 2% or 1% of the food composition. Any minimum and maximum can be combined to form a range, with the proviso that the minimum amount is less than the maximum amount in the range. Thus, the amount may, for example, be between 0.1% w/w and 15% w/w or between 0.1% w/w and 5% w/w, as non-limiting examples.

In embodiments, the food composition comprises a honey product in an amount the range of about 10 to 99 wt %, based on the total weight of the food composition.

In embodiments, the honey product is present at about 10 wt %, about 15 wt %, about 20 wt %, about 25 wt %, about 30 wt %, about 35 wt %, about 40 wt %, about 45 wt %, about 50 wt %, about 55 wt %, about 60 wt %, about 65 wt %, about 70 wt %, about 75 wt %, about 80 wt %, about 85 wt %, about 90 wt %, about 95 wt % or about 99 wt %, based on the total weight of the composition or medicament.

The food composition may comprise any other edible components. In some embodiments, the food composition is in the form of a concentrated food composition comprising a minimum of 50% (w/w) of the combination of Leptospermum extract and honey product. The amount may be a minimum of 60%, 70%, 80%, 90% or 95% of the food composition.

In some embodiments, the composition further comprises one or more of: suspending agents, emulsifying agents, gelling agents, wetting agents, fillers, binders, preservatives, flavouring, humectants, colouring and/or sweetening agents.

The aqueous extracts of the present invention may be administered as recommended by the subject's primary clinician, for example daily, every two days, three times per week, or every week, two weeks, three weeks, monthly, once every two months, or at a greater interval. The aqueous extracts may also be administered once or more daily, weekly, monthly or yearly.

A subject may be administered 1, 2, 3, 4, 5, 6, 7, 8, 9, or 10 doses of the aqueous extract.

While the exact dosage of the extract to be administered is at the discretion of the subject's primary physician, example dosage amounts could be about 0.001 mg/kg to a subject in need of such treatment to about 100 mg/kg, about 0.001 mg/kg to about 50 mg/kg, about 0.001 mg/kg to about 25 mg/kg, about 0.001 mg/kg to about 10 mg/kg, about 0.001 mg/kg to about 5 mg/kg; about 0.001 mg/kg to about 1 mg/kg or about 0.001 mg/kg to about 0.01 mg/kg. In some embodiments, the dosage of aqueous extract or composition that can be used in the methods provided herein is about 0.1 mg/kg/day, about 0.2 mg/kg/day, about 0.3 mg/kg/day, about 0.4 mg/kg/day, about 0.5 mg/kg/day, about 0.6 mg/kg/day, about 0.7 mg/kg/day, about 0.8 mg/kg/day, about 0.9 mg/kg/day, about 1 mg/kg/day, about 1.5 mg/kg/day, about 2 mg/kg/day, about 2.5 mg/kg/day, about 2.75 mg/kg/day, about 3 mg/kg/day, about 4 mg/kg/day, about 5 mg/kg/day, about 6 mg/kg/day, about 6.5 mg/kg/day, about 6.75 mg/kg/day, about 7 mg/kg/day, about 7.5 mg/kg/day, about 8 mg/kg/day, about 8.5 mg/kg/day, about 9 mg/kg/day, about 10 mg/kg/day, about 11 mg/kg/day, about 12 mg/kg/day, about 13 mg/kg/day, about 14 mg/kg/day or about 15 mg/kg/day. Consistent with the above description, any amount of solvent (e.g. water or other polar solvent) is excluded when calculating the amount of extract. Expressed in alternate terms, the dosage amount of extract refers to the active content of the extract.

In embodiments comprising a food composition that comprises the extract, the amount of food composition to be administered is within the range of about 1 g to about 50 g per dosage. The dosage may be a daily dosage amount.

In embodiments, the amount of food composition to be administered is about 1 g, about 5 g, about 10 g, about 15 g, about 20 g, about 25 g, about 30 g, about 35 g, about 40 g, about 45 g, or about 50 g.

As detailed above, the inventors have also discovered an improved method of producing an extract from a plant of the genus Leptospermum, using a polar solvent to extract actives from the plant.

In one embodiment of the invention, there is provided a method of producing an extract, the method comprising:
contacting fragmented plant material with a polar solvent for a period of at least about 10 minutes to about 2 hours and at a temperature of at least 50° C. to 120° C.;
separating remaining solids from the polar solvent to yield the extract.

The method may further comprise a preliminary step of drying the fragmented plant material to a moisture content of not greater than 10%. There may additionally be a step of fragmenting the plant material, otherwise the form of plant material that is sourced may be on that is already in the form of fragments.

The plant material is fragmented in order to improve the efficacy of the drying process, by increasing the surface area of the plant material being processed. The fragmentation process may be done by any number of ways known in the art, for example, chopping, grinding, shredding, agitating, compacting, crushing or any other suitable mechanical forces. The plant material may be fragmented to a suitable size prior to the extraction process. Preferably, the plant material is fragmented to about 1 cm to about 5 cm pieces.

The term “plant material” refers to all or part of the plant which comprises bioactive components. Plant material includes, but is not limited to, leaves, stems, bark, roots, flowers, fruits, cuttings, cells, and combinations thereof. Preferably, the plant material comprises leaves and/or stems. Preferably, the plant material subjected to the extraction process comprises at least 60% by weight of leaves and/or stem material. The selection of the plant material component for use in the extract step can influence the balance of actives in the extract so produced. The plant material subjected to the extraction process may comprise at least 60%, 70%, 80% or at least 90% by weight of the leaves and/or stems of the plant. Another example of a suitable plant material is plant mulch. Plant mulch comprises leaves, branches, stems or foliage. The plant mulch is typically in a fragmented form. The plant mulch may be a plant mulch that has been prior processed, for example by distillation or steaming. In a preferred embodiment, the plant mulch is from Leptospermum scoparium (the Manuka plant).

The person skilled in the art will appreciate that the exact composition of the extract may also vary depending on the time of the year, i.e. whether it is during the growing season, harvesting season or otherwise.

In some embodiments, the fragmented plant material is dried to a moisture content not greater than 10% prior to contacting with the polar solvent.

Once fragmented, the plant material may subsequently be dried. A number of methods for drying the Leptospermum plant material are known in the art. For example, dehydration can be done by using conventional methods, such as hot air drying, or vacuum drying. Alternatively, dehydration can be also carried out using advanced methods, such as freeze-drying or microwave drying. The exact method used may be varied depending on the exact commercial circumstances, for example cost-effectiveness and other manufacturing considerations.

Leptospermum leaves and small stems have a moisture content of over 55%, therefore dehydration or removal of the water in fresh leaves and stems decreases enzymatic activity of enzymes such as polyphenol oxidase, which could degrade the active components, and to prevent microbial spoilage. Further, dehydration of the plant material may provide the advantages of minimising costs for storage and transportation. However, the exact dehydration conditions and methods used can affect the retention of bioactive compounds and subsequently the therapeutic activity of the extracts. Further, the extracts can contain a large number of uncharacterised bioactive compounds, for which their stability to heat is not well understood.

In embodiments, the fragmented plant material is dried using hot air drying.

In embodiments, the fragmented plant material is dried using hot air drying at a temperature of about 50° C. to about 120° C.

In embodiments, the fragmented plant material is dried at a temperature of about 50° C., about 55° C., about 60° C., about 65° C., about 70° C., about 75° C., about 80° C., about 85° C., about 90° C., about 95° C., about 100° C., about 105° C., about 110° C., about 115° C., or about 120° C.

In embodiments, the fragmented plant material is dried for a period of about 15 minutes to 90 minutes.

In embodiments, the fragmented plant material is dried for a period of about 25 minutes, about 30 minutes, about 35 minutes, about 40 minutes, about 45 minutes, about 50 minutes, about 55 minutes, about an hour, about 65 minutes, about 70 minutes, about 75 minutes, about 80 minutes, about 85 minutes, or about 90 minutes.

In embodiments, the fragmented plant material is dried using vacuum drying.

In embodiments, the fragmented plant material is dried using vacuum drying using a gauge vacuum pressure in a range between about 10-100 kPa.

In embodiments, the fragmented plant material is dried using vacuum drying using a gauge vacuum pressure set at about 10 kPa, about 15 kPa, about 20 kPa, about 25 kPa, about 30 kPa, about 35 kPa, about 40 kPa, about 45 kPa, about 50 kPa, about 55 kPa, about 60 kPa, about 65 kPa, about 70 kPa, about 75 kPa, about 80 kPa, about 85 kPa, about 90 kPa, about 95 kPa, or about 100 kPa.

In embodiments, the Leptospermum plant material after drying has a moisture content of not greater than 15%. The moisture content after drying may be not greater than 14%, 13%, 12%, 11%, 10%, 9%, 8%, 7%, 6% or 5%.

In embodiments, the Leptospermum plant material after drying has a moisture content of about 1%, about 2%, about 3%, about 4%, about 5%, about 6%, about 7%, about 8%, about 9%, or about 10%.

In embodiments, the Leptospermum plant material after drying has a water activity of not greater than 0.6.

In embodiments, the Leptospermum plant material after drying has a water activity of about 0.1, about 0.2, about 0.3, about 0.4, about 0.5, or about 0.6.

Various methods and equipment are known in the art for measuring moisture content and water activity. For example, an infrared moisture balance or a water activity meter may be used to determine water activity.

If a drying process is utilised, then following the drying process the fragmented dried plant material may be further processed. For example, the particle sizes of the fragmented dried plant material may be further reduced to improve the efficiency of downstream steps.

In embodiments, the fragmented dried plant material is subjected to grinding.

In embodiments, the fragmented dried plant material is reduced to a particle size of not greater than about 2000 μm. Particle sizes are determined by reference to the size of particles that pass through a mesh of the given size. Preferably 95% or 100% of particles pass the relevant mesh size.

The particle size may be greater than 100 μm. That is, the particles that pass a 100 μm size mesh are removed, such that the particles have a size greater than 100 μm (based on 100 μm mesh size).

In embodiments, the fragmented dried plant material is reduced to a particle size of not greater than about 100 μm, not greater than about 150 μm, not greater than about 200 μm, not greater than about 250 μm, not greater than about 500 μm, not greater than about 800 μm, not greater than about 1000 μm, not greater than about 1500 μm, not greater than about 1800 μm, or not greater than about 2000 μm.

Various methods and equipment are known in the art for reducing the particle sizes of the fragmented dried material. Example equipment suitable for this step are grinding or milling machines. Preferably, a grinding machine is used. When a grinding machine is used, mesh sizes of about 50 μm to about 2000 μm are used. In embodiments, the mesh size used is about 50 μm, about 100 μm, about 150 μm, about 200 μm, about 250 μm, about 500 μm, about 800 μm, about 1000 μm, about 1500 μm, about 1800 μm, or about 2000 μm. The particle sizes may be such that they pass a mesh with an upper mesh size based on one of these values, but do not pass a mesh with a lower size based on another of these values, such that the particle size is within a range from a lower value to a higher value. As one example, the particle size range may be 200 μm-1500 μm based on mesh sizes. The particles are preferably 95% within the specified range, or up to 100% within the specified range.

Extraction is the next step for separating the bioactive compounds from the Leptospermum plant material. As the types and composition of bioactive compounds vary widely in Leptospermum plant materials, extraction efficiency can be affected by the extraction techniques and extraction conditions. A number of methods for extracting the bioactive compounds from the Leptospermum plant material are known in the art, including conventional and advanced processes. Examples of conventional extraction methods include, but are not limited to, decoction, infusion, agitation, or maceration, which are applied based on the diffusion of bioactive compounds from plant materials to the solvent. Examples of advanced techniques include, but are not limited to, ultrasonic-assisted extraction, microwave-assisted extraction, supercritical fluid extraction, ultrahigh-pressure extraction, which may have advantages relating to increased extraction efficiency or shortened extraction time. A number of factors affect the extraction efficiency, for example type of solvent used, agitation conditions, pre-leaching time, extraction temperature, length of extraction, sample-to-solvent ratio, particle size of the initial plant material, and extraction pH.

The inventors have generated an extraction protocol using a polar solvent under specific controlled conditions to extract actives from Leptospermum plant material, which advantageously allows for gentler extraction of bioactive compounds, thus minimising degradation of these actives over the course of the extraction.

During this process, the polar solvent is contacted with the fragmented plant material to extract polar-solvent soluble actives from the plant material which subsequently dissolves and admixes with the polar solvent.

In embodiments, the plant material-to-solvent ratio is about 1 kg/100 L to about 20 kg/100 L. The plant material-to-solvent ratio may be about 1 kg/100 L, about 2 kg/100 L, about 3 kg/100 L, about 4 kg/100 L, about 5 kg/100 L, about 6 kg/100 L, about 7 kg/100 L, about 8 kg/100 L, about 9 kg/100 L, about 10 kg/100 L, about 11 kg/100 L, about 12 kg/100 L, about 13 kg/100 L, about 14 kg/100 L, about 15 kg/100 L, about 16 kg/100 L, about 17 kg/100 L, about 18 kg/100 L, about 19 kg/100 L, or about 20 kg/100 L.

In embodiments, continuous agitation is utilised to enhance the efficiency of the extraction process.

In embodiments, continuous agitation is provided by mechanical forces, such as with the use of shaking, stirring, wave, rotary or tumbling motions.

In embodiments where rotational or tumbling motion is applied, the rotational or tumbling motion is provided at a rate of about 50 rpm to about 800 rpm.

In embodiments, the continuous agitation is provided at a rate of about 50 rpm, about 100 rpm, about 150 rpm, about 200 rpm, about 250 rpm, about 300 rpm, about 350 rpm, about 400 rpm, about 450 rpm, about 500 rpm, about 550 rpm, about 600 rpm, about 650 rpm, about 700 rpm, about 750 rpm, or about 800 rpm.

The polar solvent is contacted with the fragmented plant material for a period of at least about 10 minutes to about 2 hours. In embodiments, the polar solvent is contacted with the fragmented plant material for a period of about 10 minutes, of about 20 minutes, about 30 minutes, about 40 minutes, about 50 minutes, about an hour, about 70 minutes, about 80 minutes, about 90 minutes, about 100 minutes, about 110 minutes or about 2 hours.

The polar solvent is contacted with the fragmented plant material at a temperature of at least 50° C. to 120° C. In embodiments, the polar solvent is contacted with the fragmented plant material at a temperature of about 50° C., about 60° C., about 70° C., about 80° C., about 90° C., about 100° C., about 110° C., or about 120° C.

In embodiments, at least about 60% of the bioactive compounds are extracted from the plant material. For example, if the plant material contains 1 g/kg of bioactive compounds, then the methods of extraction according to the present invention recovers at least 60% of the total bioactive compounds, i.e. approximately 0.6 g/kg.

In embodiments, about 60%, about 65%, about 70%, about 75%, about 80%, about 85%, about 90%, about 95%, or about 100%, of the bioactive compounds are extracted from the plant material.

In the final step, the remaining solids are removed from the polar solvent, which at this point contains the actives, to yield the extract.

In embodiments, the extract contains at least 1% of extractable solid content.

In a preferred embodiment, the polar solvent is water.

In most scenarios, the polar solvent used is likely to be water. The use of water is advantageous because it is inexpensive, accessible, and safe, thus it is suitable for further application of the extracts in pharmaceutical and food products, or safe for human consumption.

In embodiments, the method of the present aspect comprises removing or reducing the amount of polar solvent following separation of the solids, to yield a concentrated extract. Alternatively, or in addition to removing or reducing the amount of polar solvent, the extract or concentrated extract can be further fractionated to separate the extract or concentrated extract into portions comprising a specific number of compounds. Any number of methods known in the art are suitable for removing or reducing the polar solvent following separation of the solids, for example by filtration, centrifugation, evaporation, condensation, chromatography, crystallization, liberation and lyophilisation. Preferably, the polar solvent is removed using evaporation and condensation.

In embodiments, the polar solvent may also be removed following separation of the solids to yield a dry extract.

In embodiments, the polar solvent is removed under vacuum.

In some embodiments, the polar solvent is removed under vacuum at a pressure of about 1 kPa to about 20 kPa.

In some embodiments, the polar solvent is removed under vacuum at a pressure of about 1 kPa, about 2 kPa, about 3 kPa, about 4 kPa, about 5 kPa, about 6 kPa, about 7 kPa, about 8 kPa, about 9 kPa, about 10 kPa, about 11 kPa, about 12 kPa, about 13 kPa, about 14 kPa, about 15 kPa, about 16 kPa, about 17 kPa, about 18 kPa, about 19 kPa, or about 20 kPa.

Preferably, the vacuum pressure is selected from the group consisting of: about 2.3 kPa, about 4.2 kPa, about 7.3 kPa, 12.3 kPa, and about 20 kPa.

The polar solvent is removed at a temperature of between about 20° C. to about 60° C. In embodiments, the polar solvent is removed at a temperature of about 20° C., about 30° C., about 40° C., about 50° C., or about 60° C.

In embodiments, the aqueous extraction is performed under vacuum.

In embodiments, the methods of the present aspect further comprise the step of encapsulating the extract to form an encapsulated extract.

As detailed previously, the encapsulation process protects the bioactive compounds from degradation. The encapsulation process is thus thought to improve bioaccessibility and bioavailability by coating the active component with an encapsulating agent and providing a layer of protection, and may also assist with targeted delivery to specified parts of the gastrointestinal tract where uptake is most desirable, and/or allows for controlled release of the active component. This is particularly the case when the extract is in powder form, where the stability of the bioactive compounds is very low due to the increased surface area resulting in increased exposure to the environment, as well as the high hygroscopicity of the powder.

The present disclosure encompasses various modifications to encapsulation processes known in the art, for example varying the ratio of encapsulating material-to-extract, temperature, such as inlet temperature, outlet temperature, vacuum pressure, aspiration rate, and feeding rate in order to obtain encapsulated extract with good physical properties (for example colour, moisture, water activity, hygroscopicity, bulk density, followability and solubility), good phytochemical properties (for example presence of compounds such as phenolic compounds and MGO), potent antioxidant properties and biological properties.

Various methods and equipment for performing the encapsulation process are known in the art. Preferably, the encapsulation process is conducted using spray drying, vacuum drying or the freeze drying techniques.

The water content of the extract may be reduced prior to mixing with the encapsulation agent. This may be achieved by evaporation (e.g. partial evaporation). The water content may be reduced by heating the extract, or exposing the extract to vacuum pressure.

In embodiments, the extract is heated to a temperature between about 30° C. to about 120° C. In embodiments, the extract is heated to a temperature between about 40° C. to about 100° C. In embodiments, the extract is heated to a temperature between about 40° C. to about 80° C. In embodiments, the extract is heated to a temperature between about 40° C. to about 60° C. In embodiments, the extract is heated to a temperature between about 50° C. to about 100° C. In embodiments, the extract is heated to a temperature between about 50° C. to about 80° C. In embodiments, the extract is heated to a temperature between about 60° C. to about 100° C. In embodiments, the extract is heated to a temperature between about 60° C. to about 80° C.

In embodiments, the extract is heated to a temperature of about 30° C., about 40° C., about 50° C., about 60° C., about 70° C., about 80° C., about 90° C., about 100° C., about 110° C., or about 120° C.

In embodiments, the water content of the extract is reduced by exposure to a pressure between about 10 kPa to about 60 kPa. In embodiments, the extract is exposed to a pressure between about 20 kPa to about 50 kPa. In embodiments, the extract is exposed to a pressure between about 30 kPa to about 40 kPa. In embodiments, the extract is exposed to a pressure between about 30 kPa to about 50 kPa.

In embodiments, the extract is exposed to a pressure of about 10 kPa, about 15 kPa, about 20 kPa, about 25 kPa, about 30 kPa, about 35 kPa, about 40 kPa, about 45 kPa, about 50 kPa, about 55 kPa, or about 60 kPa.

In embodiments, the partially evaporated extract has a solid content of between about 1% to about 30%. In embodiments, the partially evaporated extract has a solid content of between about 5% to about 20%. In embodiments, the partially evaporated extract has a solid content of between about 5% to about 15%. In embodiments, the partially evaporated extract has a solid content of between about 10% to about 30%. In embodiments, the partially evaporated extract has a solid content of between about 15% to about 30%. In embodiments, the partially evaporated extract has a solid content of between about 15% to about 20%. Following from this, it will be understood that the water content reduction step effects a reduction of the water content of the extract to a water content within the range of about 70% to 99% (this correlates to the indicated solids content of 1% to 30%). The water content ranges for other embodiments of the evaporated extract can be calculated from the indicated ranges for the solids content values.

The partially evaporated extract, which may also be referred to as a concentrated extract, may then be mixed with one or more encapsulating agents.

In embodiments, the one or more encapsulating agents is selected from the group consisting of: Gum Arabic, Carrageenan, starch, modified starch, gluten, protein isolate, soy protein isolate, casein, whey protein, maltodextrins, ginger or coconut extracts.

In embodiments, the concentrated extract is mixed with the one or more encapsulating agents at a ratio between about 10:1 to about 1:2 (w/w). In embodiments, the concentrated extract is mixed with the one or more encapsulating agents at a ratio between about 5:1 to about 1:1 (w/w). In embodiments, the concentrated extract is mixed with the one or more encapsulating agents at a ratio between about 3:1 to about 1:1 (w/w).

In embodiments, the concentrated extract is mixed with the one or more encapsulating agents at a ratio of about 10:1, about 9:1, about 8:1, about 7:1, about 6:1, about 5:1, about 4:1, about 3:1, about 2:1, about 1:1, or about 1:2.

In embodiments, the encapsulated extract is homogenised. In embodiments, the encapsulated extract is homogenised prior to spray drying or vacuum drying.

In embodiments utilising spray drying, the feeding rate is from about 30% to 100%.

In embodiments utilising spray drying, the feeding rate is about 30%, about 35%, about 40%, about 45%, about 50%, about 55%, about 60%, about 65%, about 70%, about 75%, about 80%, about 85%, about 90%, about 90%, about 95% or about 100%.

In embodiments utilising spray drying, the inlet temperature ranges from about 100° C. to about 250° C. In embodiments utilising spray drying, the inlet temperature ranges from about 120° C. to about 200° C. In embodiments utilising spray drying, the inlet temperature ranges from about 150° C. to about 180° C. In embodiments utilising spray drying, the inlet temperature ranges from about 120° C. to about 250° C. In embodiments utilising spray drying, the inlet temperature ranges from about 120° C. to about 180° C. In embodiments utilising spray drying, the inlet temperature ranges from about 120° C. to about 150° C. In embodiments utilising spray drying, the inlet temperature ranges from about 150° C. to about 250° C. In embodiments utilising spray drying, the inlet temperature ranges from about 180° C. to about 200° C. In embodiments utilising spray drying, the inlet temperature is about 120° C., about 130° C., about 140° C., about 150° C., about 160° C., about 170° C., about 180° C., about 190° C., about 200° C., about 210° C., about 220° C., about 230° C., about 240° C., or about 250° C.

In embodiments utilising vacuum drying, the temperature is set to at least 50° C. to 120° C. In embodiments, the polar solvent is contacted with the fragmented plant material at a temperature of about 50° C., about 60° C., about 70° C., about 80° C., about 90° C., about 100° C., about 110° C., or about 120° C.

In embodiments utilising vacuum drying, the vacuum pressure is set from a range of about 10 kPa to about 60 kPa.

In embodiments utilising vacuum drying, the vacuum pressure is set at about 10 kPa, about 15 kPa, about 20 kPa, about 25 kPa, about 30 kPa, about 35 kPa, about 40 kPa, about 45 kPa, about 50 kPa, about 55 kPa, or about 60 kPa.

It must also be noted that, as used in the specification and the appended claims, the singular forms ‘a’, ‘an’ and ‘the’ include plural referents unless otherwise specified. Thus, for example, reference to ‘bioactive compound’ may include more than one bioactive compound, and the like.

Throughout this specification, use of the terms ‘comprises’ or ‘comprising’ or grammatical variations thereon shall be taken to specify the presence of stated features, integers, steps or components but does not preclude the presence or addition of one or more other features, integers, steps, components or groups thereof not specifically mentioned.

Unless specifically stated or obvious from context, as used herein, the term ‘about’ is understood as within a range of normal tolerance in the art, for example within two standard deviations of the mean. ‘About’ can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein in the specification and the claim can be modified by the term ‘about’.

Any methods provided herein can be combined with one or more of any of the other methods provided herein.

Ranges provided herein are understood to be shorthand for all of the values within the range. For example, a range of 1 to 50 is understood to include any number, combination of numbers, from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50. Further, all ranges provided herein also encompass any and all possible sub-ranges and combinations of sub-ranges thereof. For example, a range of 1 to 10 is understood to include the sub-ranges of 1 to 5, 1 to 10, 5 to 10, 4 to 7, and so on. Language such as ‘up to’, ‘at least’, ‘greater than’, ‘less than’ and the like include the number recited and refer to ranges which can subsequently be broken down into sub-range as discussed above. Finally, ranges are to be interpreted as also including each individual integer of the range.

Reference will now be made in detail to exemplary embodiments of the disclosure. It is understood that the detailed examples and embodiments described herein are given by way of example for illustrative purposes only, and are not to be considered to be limiting to the disclosure.

### EXAMPLES

### Example 1. Methods and Materials

### Animal Model and Cell Lines

The study utilised severe combined immunodeficiency (SCID) mice inoculated with human mesothelioma cancer cell lines (MSTO cells) and human lung cancer cell lines (H1975 cells). Human mesothelioma cell lines (H2052, H2452 and MSTO) and lung cancer cell lines (H1975 and H3122) were purchased from American Type Culture Collection (ATCC).

For the cancer cell proliferation assays, the same human mesothelioma cell lines used for inoculation in the SCID mice were used, and additionally primary malignant pleural mesothelioma (MPM) cell lines (MM05 and Ren) were also included. All cell lines were cultured in RPMI-1640 medium with 10% foetal bovine serum (FBS), and maintained at 5% CO₂, 37° C. and 95% humidity.

### Drug Treatment and AlamarBlue® Proliferation Assay

alamarBlue® cell death assays were carried out for cells as follows: Cells were plated in 96-well culture plates at 2 500 cells in 100 μl medium per well. After 24 hr, cells were treated with LEP at various concentrations, and then incubation of cells continued for 72 hr. AlamarBlue®, 15 μl (50 mL PBS containing also Sigma reagents 0.075 g Resazurin, 0.0125 g Methylene Blue, 0.1655 g Potassium hexacyanoferrate (III), 0.211 g Potassium hexacyanoferrate (II) trihydrate, filter-sterilised, and stored at 4° C. in the dark), were added and incubated for 4 hr at 37° C. Fluorescence intensity was measured at 590-10 nm with 544 nm excitation, using a FLUOstar Optima (BMG LabTech, Ortenberg, Germany). Fluorescence intensity was calculated as a percentage of intensity of control cells (untreated). Experiments involving human cell lines were performed three times with three replicates per repeat. All media and FBS were from Life Technologies (Carlsbad, CA, USA).

### Cell Migration Assay

Cell migration of the V40 cancer cell line was measured using a scratch (wound-healing) assay. Briefly, cells were plated in 24-well plates and 24 h post-seeding, 10 μg/mL camptothecin (Sigma-Aldrich) was added to stop cell proliferation; at the same time, a cross-shaped scratch was made using a 200 μl plastic pipette tip. At 12, 24 hr post scratch, microscopic imaging was taken with a 20× objective (Leica). Each experiment group was performed in duplicate.

### Seahorse Extracellular Flux Analysis

The seahorse XF24 Extracellular Flux Analyzer (Agilent, CA, USA) was used to measure the respiration activity of the MSTO mesothelioma cancer cell line. Cells were seeded at the density of 8×10⁴ cells per well in a XF24 plate overnight and treated with and without QV0 for 48 hrs. The mitochondrial stress test was performed according to manufacturer's instruction. Briefly, 1 μM oligomycin (oligo), 0.3 μM FCCP, and 1 μM Rotenone and Antimycin A were added and the relative levels of basal, maximal respiration and reserved mitochondrial capacity were calculated based on OCR data obtained in the Mito stress tests using Seahorse Wave software for XF analyzers.

### Animal Experiments

All animal experiments were approved by the Sydney Local Health District Animal Welfare Committee. MSTO-211H (1.0×10⁶) cells and H1975 (1.0×10⁶) cells were injected intraperitoneally into 10-week-old female SCID mice. Ten SCID mice (10 weeks old, female) were separated into two groups. The Lep treated group were daily oral feed with 5 mg/20 g Lep extracts from 7 days prior to the experiment. The control group were fed with PBS as control. The tumours were visualized with the IVIS spectrum in vivo imaging system on day 9, 16, 23 and 30.

### Example 2. Production of Leptospermum Extract

The inventors disclose herein example conditions for (i) dehydration of Leptospermum plant materials using vacuum and hot air oven drying techniques; (ii) aqueous extraction of bioactive compounds from Leptospermum plant materials; and (iii) encapsulation of Leptospermum extract enriched with bioactive compounds.

### (i) Dehydration of Leptospermum Plant Materials

### 1. Harvest:

The leaves and small stems harvested from the trees manually or using a machine. Harvesting must be done on the day of drying or dehydration, and the leaves and small stems should be free of dirt and insects. After harvesting, the leaves and stems are transported to the factory for drying as soon as possible. If drying cannot be done within 3 days, the leaves and stems are preferably stored at 2-5° C. to minimize oxidation. Before drying, the leaves and small stems may additionally be fragmented into smaller sizes ranging from 1.5 to 5 cm to enhance the effectiveness of the dehydration process.

### 2. Dehydration:

The leaves and stems are then subjected to hot-air drying or vacuum drying for removal of water. Drying can be done in batch or continuous processing. For hot-air drying, leaves and stems are separated on a tray or belt with a layer thickness of 1-3 cm. Air ventilation with temperature is set from 70-100° C. for approximately 25 min-1 hour. The drying time may be varied depending on the temperature applied. As a general rule, the higher the temperature, the less drying time is required. Similarly, for vacuum drying the leaves and stems are subjected to vacuum drying, set at gauge vacuum pressure ranging from 20-60 kPa, temperature 70-100° C., for a total drying time of approximately 25 min-1 hour. Drying time is dependent on vacuum pressure and temperature. Dried leaves have a moisture content of below 8% and water activity of below 0.5.
   3. Packaging and storage:
After drying is completed, the dried leaves and stems are ground into small particle sizes, in this example using 250 μm mesh on a grinding machine. The ground materials are then packed in a plastic bag (polyethylene) or container, preferably in vacuum packing. The bag and container must keep the ground materials from moisture, light and other odors. The materials are then stored at room temperature for further processing.
   (ii) Aqueous Extraction of Bioactive Compounds from Leptospermum Plant Materials

### 1. Extraction:

Water is heated to the determined temperature ranging from between 70-100° C. The ground dried leaves and small stems are then added at a sample-to-solvent ratio of 4-10 kgs/100 L. Continuous agitation (100-600 rpm) is applied to support the extraction process. Temperature is maintained and extraction is conducted for 30 min-1 hour to extract over 80% of bioactive compounds from the materials. After completion of the extraction, the extract is quickly cooled down to room temperature to minimize degradation.

### 2. Separation of the Solids in the Extract:

The remaining spent leaves and stems in the extract are separated using filtration or centrifugation technique. Sieves and or membranes can also be applied to separate the remaining leaves and stems. The extract should contain more than 1% of extractable solid content.

### 3. Evaporation and Condensation:

As the extract contains a high level of water, evaporation is applied to partially remove the water from the extract and increase the extractable solid content which includes the bioactive compounds. In this example, evaporation and condensation techniques are utilized. Temperature is set in a range of 30-50° C. to minimise the degradation of the bioactive compounds. Vacuum pressure is set to a value within the range of about 4.2 kPa to about 12.3 kPa to evaporate water from the extract. The vapour is condensed, and water is discarded. Extract with solid contents of above 5% are obtained for further processing. The extract can be stored at room temperature if further processing is conducted within 3 days. It should be stored at 5° C. if it is processed with a week and at −18° C. if it is processed over a week.
   (iii) Encapsulation of Leptospermum Extract

### 1. Addition of Encapsulating Materials:

Various encapsulating materials, such as carbohydrate polymers (starch, gums, alginate, carrageenan), and proteins (gluten, protein isolate, casein, whey protein) can be applied to encapsulate the Leptospermum extract. The encapsulating materials can be applied individually or in combination to the extract. The incorporation process can be done by adding the encapsulating agents directly to the Leptospermum extract or mixing them in water to obtain a solid content ranging from 3% to 30%. The mixture is then heated up and then homogenized for the next step.

### 2. Encapsulation Process:

Encapsulation can be conducted using either spray drying, vacuum drying or freeze drying technique. Example conditions are provided as follows: for spray drying, the feeding rate ranges from 30-100%, air flow is set corresponding to the feeding rate, inlet temperature ranges from 140-180° C. For freeze drying, the mixture is frozen before freeze drying to obtain the dried extract. Dried extract is then ground into fine powder using a grinding machine. For vacuum drying, temperature is setting from 60-100° C. with vacuum pressure ranging from 10-40 kPa and drying is conducted to obtain the dried extract. Dried extract is then ground into fine powder using a grinding machine.
An exemplary evaporation and encapsulation protocol is as follows:
   The extract is partially evaporated using reduced vacuum pressure and temperature ranging from 40-80° C. to obtain an extract with the solid content of 5-15%.
   The concentrated extract is then mixed with one or combined of the encapsulating materials, such as Gum Arabic, Carrageenan, modified starch, soy protein isolate, maltodextrins, ginger or coconut extracts at a ratio ranging from 0.2-1 (w/w). The mixture is homogenised using a commercial mixer.
   The homogenised mixture is then spray dried set at inlet temperature ranging from 120-200° C. to obtain the encapsulated extract powder.

### 3. Packaging and Storage:

Powders are then packed in a plastic bag (polyethylene) or container, preferably vacuum packing. The bag and container must protect the powders from moisture, light and other odors. Powders are then stored at room temperature for further processing.

### Example 3. Use of Leptospermum Extract in Inhibiting Cancer Cell Proliferation In Vitro

### Results

### Leptospermum (Lep) Extracts Inhibit Cell Proliferation in Various Cancer Lines.

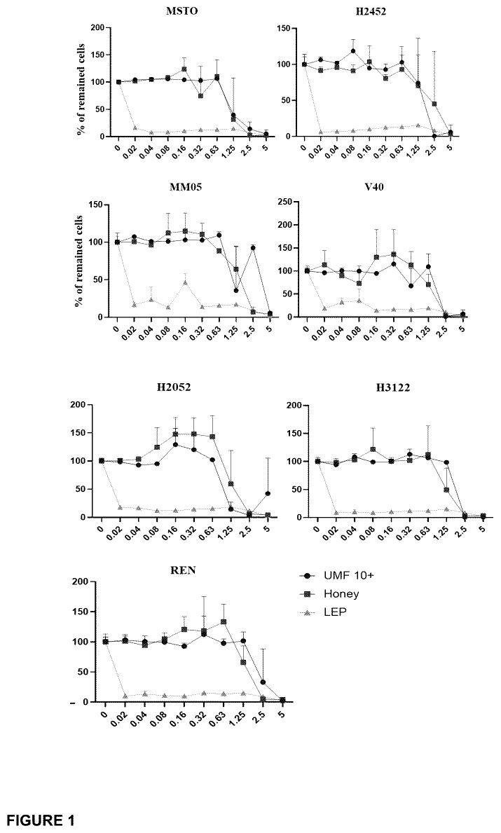

The effect of Lep extracts on cancer cell lines was assessed using a cytotoxic assay (cell proliferation), and compared with UMF10+ Manuka Honey (Comvita), and Honey formula (90% Manuka, 7.5% Orange peel, 2.5% Lep) as controls. Lep extracts were shown to suppress mesothelioma and lung cancer cell line growth significantly when compared to controls (FIG. 1). Further, the anti-tumoural response of Lep is not dose dependent, with cancer killing activity present at even the lowest concentrations for the Lep extract.

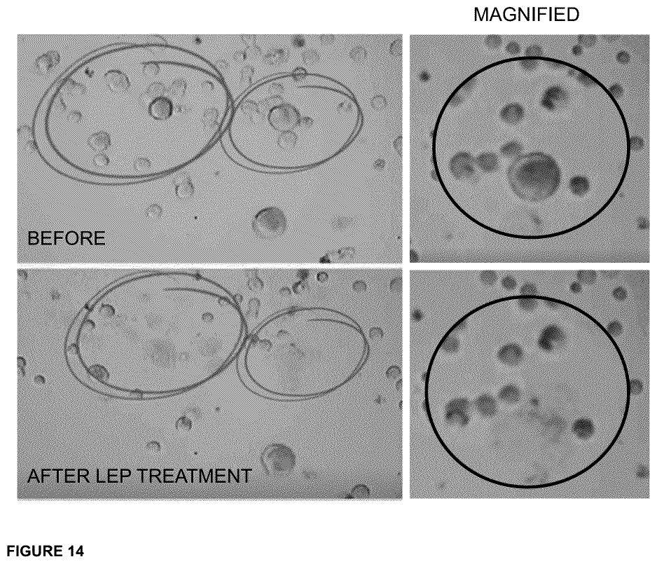

The effect of Lep extracts on the V40 cancer cell line was shown by timelapse recording over a period of 24 hours. As shown in FIG. 14, cancer cells exposed to Lep extracts underwent cell death, which supports the anti-cancer properties of the extracts.

### Lep Extract Inhibits Cell Migration and Increased Mitochondrial Spare Respiratory Capacity in Cancer Cells.

The effect of Lep extract on cancer cell migration was assessed using the scratch (wound-healing) assay. Cancer cell lines were shown to exhibit reduced cell migration after treatment with the Lep extract, as shown in Table 1 below.

TABLE 1
Results of the cell migration assay

| | | Migration at | Migration at | Distance of |
|---|---|---|---|---|
| | Cell line | 0 HR (μm) | 24 HR (μm) | migration (μm) |
| Control | V40 | 300 | 100 | 200 |
| Lep extract | V40 | 400 | 300 | 100 |

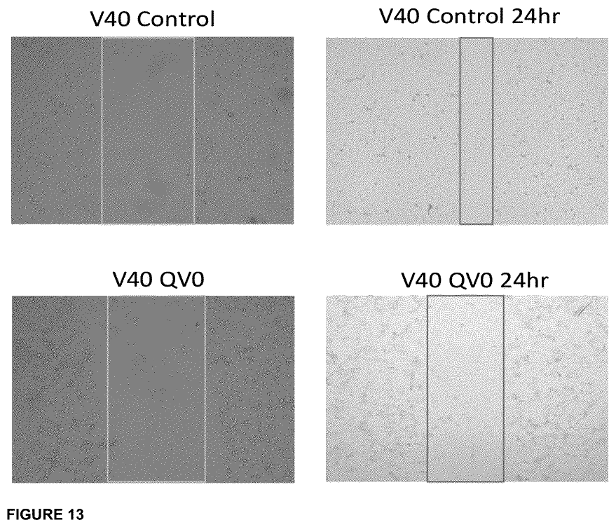

As illustrated in FIG. 13, cells from the V40 cancer cell line treated with the Lep extracts exhibited reduced cell migration as compared to controls. The data suggests that treatment with the Lep extracts could reduce invasion of cancer cells throughout the organs of cancer patients, and may also reduce the rates of metastasis in these patients.

Next, the effect of Lep extract on the mitochondrial function and oxygen consumption rate of the MSTO mesothelioma cancer cell line was assessed using the Seahorse XF Cell Mito Stress Test (data not shown). The maximal respiration of MSTO cells was increased after treatment with Lep extract as compared to the human fibroblast controls. Moreover, mitochondrial spare respiratory capacity was also specifically increased in mesothelioma cancer cells as compared to fibroblast controls, after treatment of both groups with Lep extract (control: 100 pmol/min, MSTO: 650 pmol/min). The results suggest that treatment with the Lep extract specifically increases cellular energy demand in mesothelioma cells, meaning more ATP is required in the cells to maintain normal cell function.

Without being bound to theory, the inventors envisage that treatment of Lep extracts in normal cells provides an increased energy expenditure resulting in increased metabolism, causing improved weight loss and reduction in overall blood glucose. However, cancer cells already exhibit heightened energy expenditure due to their cancerous state. Thus the treatment of cancer cells with Lep extracts results in an even further increase in energy expenditure, causing an excessively high overall energy expenditure that cannot be sustained, resulting in cancer cell death. This proposed mechanism of action supports the observation that Lep extracts specifically result in cell death of cancer cells, but provide for improved health benefits in normal cells.

### Example 4. Use of Leptospermum Extract in Suppressing Cancer Cell Growth In Vivo

### Results

### Lep Extracts Suppress the Human Mesothelioma Cancer Cell (MSTO) and Lung Cancer Cell (H1975) Growth in Severe Combined Immunodeficiency (SCID) Mice Model.

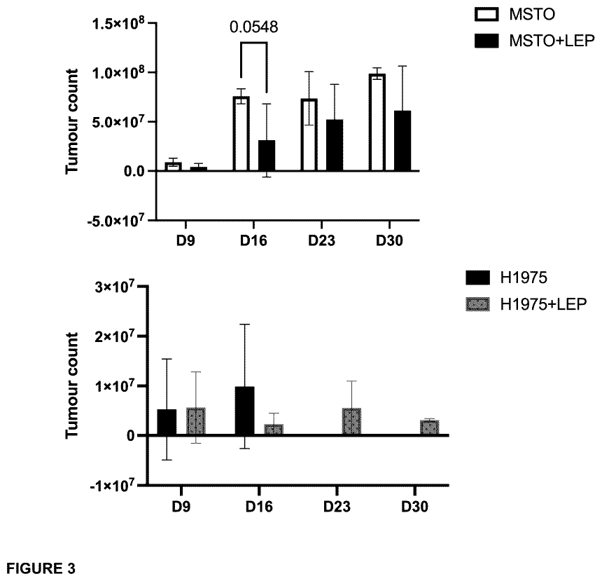

The Lep treated mice demonstrated a reduced tumour size and density over 30 days when compared to that of the control mice. This is further confirmed by quantifying the counts of bioluminescence detection (cells per second) which showed a significant reduction in the tumour cell counts observed in Lep treated mice at day 16 (FIG. 3). This effect was seen for both groups of mice that were injected with the MSTO and H1975 cells.

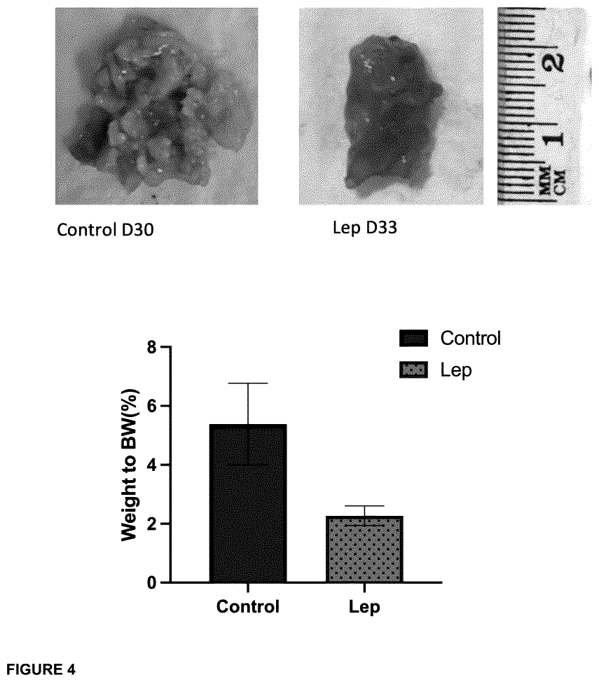

Further, the average life span of Lep treated mice that were injected with MSTO cells was extended by 3 days (33 days in Lep treated group vs 30 days in control) and the weight of the tumour mass was reduced by 4% of bodyweight with Lep treatment (FIG. 4).

The average life span of Lep treated mice that were injected with H1975 cells was extended by 14 days (30 days in Lep treated group vs 16 days in control, FIG. 3). At 16 days, the LEP treated group exhibited more than a 70% reduction in tumour cell counts as compared to the control group.

### Lep Extracts Better Maintained Body Weight in the MSTO Syngraft Model.

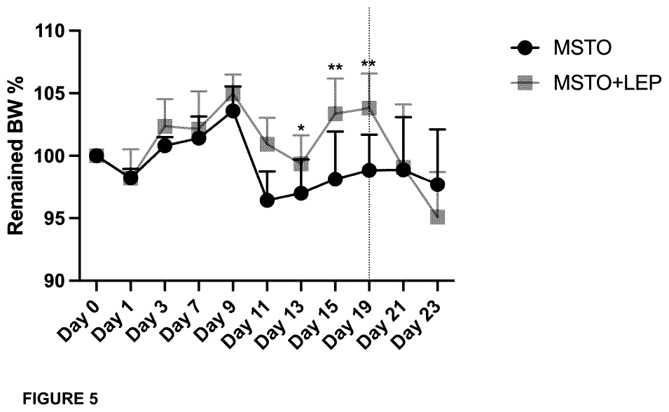

In both animals and humans undergoing cancer treatment, maintenance of body weight is an important index as a measure of general health. Mice treated with LEP extracts exhibited a better weight maintenance when compared with animals without LEP treatment (FIG. 5).

### Summary

Lep extracts exhibited anti-tumour function both in vitro and in vivo. Further, mice fed with Lep extracts had improved general health when compared to untreated mice, based on a maintenance of body weight over a 30 day period.

### Example 5. Method for Producing a Fortified Leptospermum Extract with

Leptospermum honey powder The inventors sought to apply the Lep extracts with Leptospermum honey powder in order to assess any improvements to the therapeutic utility. Leptospermum honey, which is different from regular honey, has been used by Australian Aboriginals and Maori for thousands of years for its believed medicinal properties, such as treatment of burns, cataracts, ulcers and wound healing. The medicinal properties of Leptospermum honey are largely thought to be due to the presence of methylglyoxal (MGO), which is produced non-enzymatically from dihydroxyacetone (DHA) present in nectar of Leptospermum species.

Liquid honey has a high density and viscosity, thus limiting the opportunity for combined application with other products, whereas honey powder has a wider range of opportunities for alternate applications due to its extended shelf life, making it more convenient for transportation, distribution, packaging and storage. It is also easier to apply powdered honey into cosmetic, pharmaceutical or food products. Citrus peel is known to have a high fibre content, other bioactive compounds and pectin. Provided herein is a method to produce Leptospermum honey powder fortified with the Leptospermum extract and additionally with citrus peel as a functional ingredient for further applications.

### 1. Preparation of Major Ingredients:

Citrus pomace generated from juice factories is dried using hot air oven at 70-100° C. for 5-8 hours, or vacuum drying at 70-100° C., 20-60 kPa, for 5-8 hours. After drying is completed, the dried peels are ground through less than 250 μm mesh using a grinding machine. The dried ground peels are packed in a plastic bag or container, which protect them from light, oxygen and moisture absorption. Encapsulating materials, such as carbohydrate polymers (starch, gums, alginate, carrageenan), and proteins (gluten, protein isolate, casein, whey protein) are then used to further protect the powder. Leptospermum honey with 30 plus certified MGO is used for further production of the honey powder.

### 2. Processing:

Leptospermum extract (2.5-10% of total weight), citrus peel (20-25%), and the other encapsulating materials (2.5-5%) are mixed with the Leptospermum honey. The mixture is then vacuum dried at first 25-35° C. to heat up the honey, then vacuum pressure is reduced to 10-40 kPa and the temperature subsequently increased to 90-100° C. for 100-120 min. The dried honey product is then ground into powder using a grinding machine.

### 3. Packaging and Storage:

The Leptospermum honey powder is packed in small bags and stored at room temperature.

### Example 6. Use of the Fortified Leptospermum Extract with Leptospermum Honey Powder in Treating a Metabolic Syndrome and Maintaining Healthy Glucose Levels

### Animal Model and Cell Lines

Two groups of C57 mice were used in this study (n=10 each group). In the control group C57 mice were maintained under normal conditions and fed with a normal diet. The treated group were feed with the fortified Leptospermum extract with Leptospermum honey powder (20 mg/mouse/day). The body weight and blood glucose of the animals were monitored during the 28 days period, with measurements made once a week. Blood glucose level was measured using OneTouch Verio Flex® meter. At the conclusion of the study, fasting blood glucose was measured, with animal placed under fasting conditions for 6 hours prior to measuring blood glucose levels. Plasma was subsequently collected for measuring AST/ALT levels (as a measure of overall liver function and health) and liver samples were collected for histological analysis. At day 28, body composition of the mice was measured using a DEXA scan.

### Results

### Body Weight and Composition

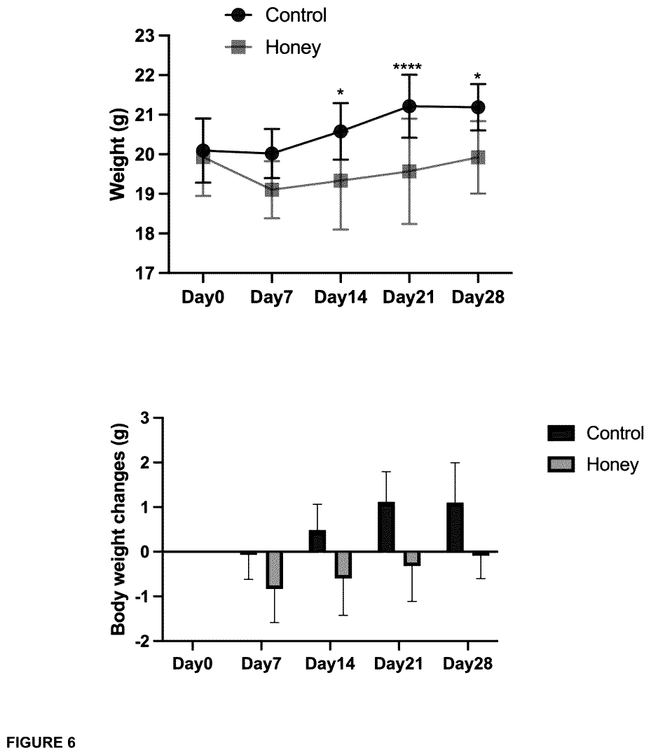

As shown in FIG. 6, the control animals were observed to gradually gain body weight over 28 days. In contrast, mice fed with the honey formula exhibited an initial body weight loss which begun at day 7 and subsequently recovered at day 28. The control animals had significant higher body weight when compared to honey fed animals at day 14, 21 and 28.

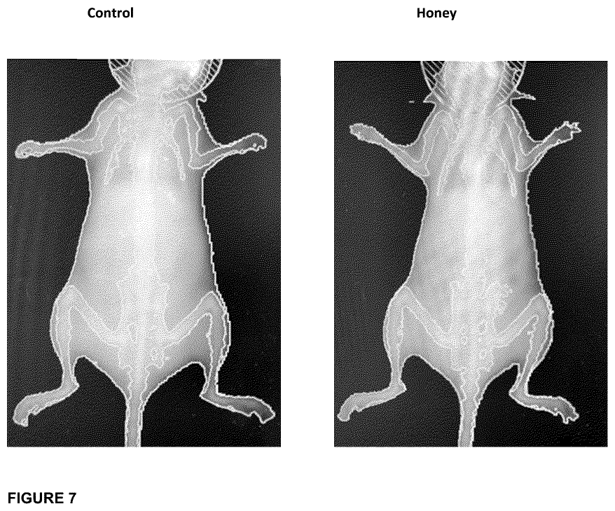

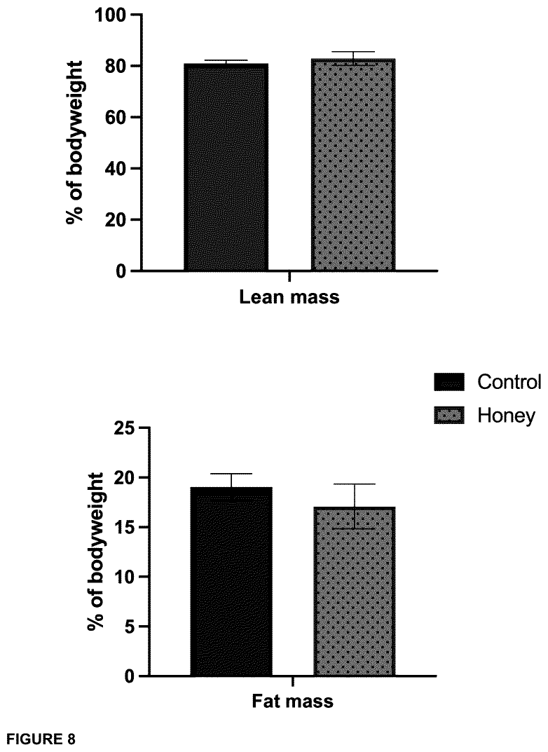

At day 28, the body composition was measured using DEXA scans (FIG. 7). The results are shown in FIG. 8. Mice fed with honey formula exhibited a reduced fat mass (16% in honey vs 19% in control) and an increase in lean mass when compared to control. Therefore, body weight loss observed in honey fed animals may associated with a reduced fat mass.

### Liver Toxicity Analysis

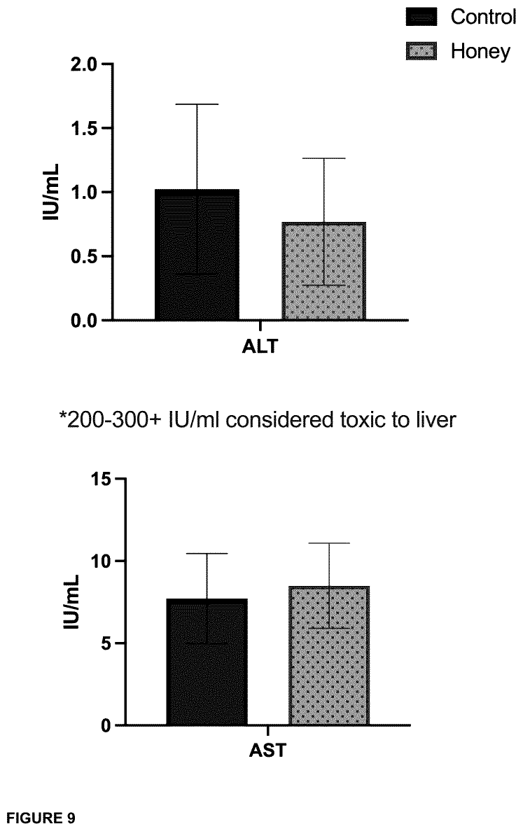

Liver toxicity was measured by quantifying the levels of liver enzymes aspartate transaminase (AST) and alanine transaminase (ALT) in the plasma of control and honey formula fed animals. Low AST and ALT concentrations were found in both groups, confirming that honey feeding did not induce liver damage. As shown in FIG. 9, AST enzyme concentrations were approximately 7-8 IU/mL in both groups, and ALT enzyme concentrations were approximately 1.0 IU/mL in the control group and 0.7-0.8 IU/mL in the honey fed group. For perspective, AST and ALT levels around 200-300+IU/mL are considered to indicate liver toxicity.

### Liver Histology Analysis

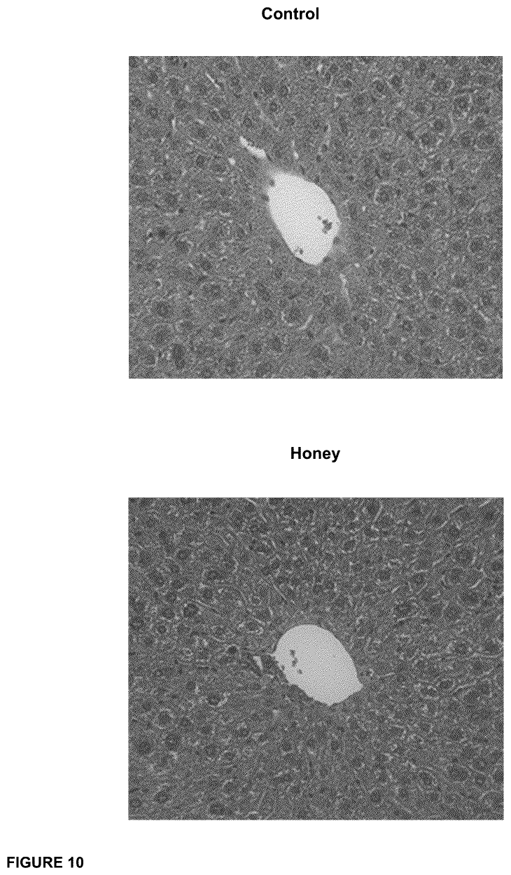

Haematoxylin and eosin (H&E) staining of day 28 liver samples was conducted on both groups of animals. As shown in FIG. 10, histological analysis revived normal liver appearance, no immune cell infiltration or fat adipocytes deposition (Fatty liver) in both groups of animals.

### Fasting Blood Glucose Concentration

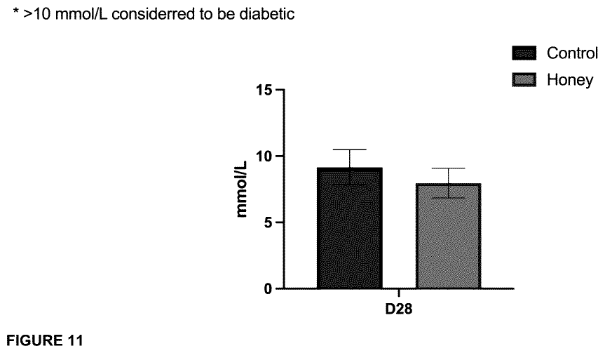

Blood sugar levels were analyzed in animals after a 6 hours fasting period prior to tail blood collection. In the honey feeding group, the fasting blood glucose was slightly lower than control animals with levels measured at 7.95 mmol/I and 9.16 mmol/I respectively (FIG. 11).

### Example 7. Analysis of the Antioxidative Capacity of the Leptospermum Extracts

### Total Phenolic Content (TPC)

The level of TPC was determined by the Folin-Ciocalteu method (AOCS, 1990) modified for microplate. Water was used as the blank, and gallic acid was used as the standard for a calibration curve. 15 μL of samples, standards (Gallic acid) and blanks (water) were placed into a 24 well microplates, followed by addition of 240 μL of diluted Folin-Ciocalteu (FC) phenol reagent (6.25%). The mixture was incubated in dark for 10 mins at room temperature followed by the addition of 15 μL of 20% sodium carbonate. The mixture was then incubated for a further 20 mins in the dark, and absorbance was measured at 765 nm using a FLUOstar Optima microplate reader (BMG LabTech, Ortenberg, Germany). The total phenolic content is calculated based on the calibration curve and expressed as milligrams of gallic acid equivalent per gram of extract.

### Scanning Major Bioactive Compounds in Leptospermum Extracts

Major phytochemicals in Leptospermum extract was determined using a Shimadzu HPLC system (Shimadzu, Japan) fitted with a reversed phase column (Luna 5u Phenyl-Hexyl 250×3.00 mm 5u micron) (Phenomenex) maintained at 35° C. by a column oven (CTO-20A, Shimadzu) with photodiode array detector (SPD-M40). The mobile phase consisted of 0.1% formic acid (Solvent A) and absolute acetonitrile (Solvent B). An auto injector (SIL-20A) was used to inject 25 μL sample volumes onto the HPLC at a flow rate of 0.7 mL/min with a gradient elution schedule as follows: 0-10 min, 0% B; 10-45 min, 40% B; 45-60 min, 60% B; 60-70 min, 60% B; 70-80 min, 0% B and 80-85 min, 0% B.

### Ferric Reducing Antioxidant Power (FRAP)

Antioxidant activity of sample was evaluated using the ferric reducing antioxidant power assay (FRAP) according to previous published method (Thaipong et al. (2006), Journal of Food Composition and Analysis) with modification for microplate. Briefly, a FRAP working solution was prepared freshly by mixing three reagents: (A) 300 mM acetate buffer (PH 3.6), (B) 10 mM TPTZ (2,4,6 tripyridyl-s-triazine) in 40 mM HCL solution and (C) 20 mM Ferric Chloride. Trolox was used as the standard for a calibration curve. 50 uL of sample, blanks (water), and standards were placed into a 24 well microplates, followed by addition of 300 uL of FRAP working solution. The mixture was incubated for 30 min at room temperature and absorbance was measured at 593 nm using a FLUOstar Optima microplate reader (BMG LabTech, Ortenberg, Germany). Antioxidant activity of plasma was expressed as milligrams of Trolox equivalent per gram of extract.

Measurement of MGO in Leptospermum Extracts Hydroxyacteone (HA) (3 mg/mL) was used as an internal standard. O-(2, 3, 4, 5, 6-pentafluorobenzyl) hydroxylamine hydrochloride (PFBHA) derivatising agent with a concentration of 10 mg/mL was prepared in 0.1M citrate buffer (pH 4). Methylglyoxal (MGO) with a concentration range from 117 to 3.656 μg/mL was prepared to generate a calibration curve. Prior to HPLC analysis, the sample solutions or MGO standards were mixed with internal standard (HA) and derivatising agent (PFBHA) in the ratio of 4:1:2 (v/v/v). The mixture was vortexed and incubated at 30° C. for 2 hours to allow for completion of derivatisation. Acetonitrile was added to each test tube in the ratio of acetonitrile to mixture of 1.7/1 (v/v) and mixed well until all crystals dissolved, then the samples or MGO standard solutions were analysed using a HPLC system. A HPLC system (Shimadzu, Japan) was fitted with a reversed phase column (Luna 5u Phenyl-Hexyl 250×3.00 mm 5u micron) (Phenomenex) maintained at 35° C. by a column oven (CTO-20A, Shimadzu) with photodiode array detector (SPD-M40). Two mobile phases were used as gradient solvents, including water: acetonitrile with the ratio of 70/30 (v/v) (Solvent A) and absolute acetonitrile (Solvent B). An auto injector (SIL-20A) was used to inject 30 μL sample volumes onto the HPLC at a flow rate of 0.8 mL/min with a gradient elution schedule as follows: 0-3 min, 0% B, 3-11 min, 90% B, 11-14 min, 90% B, 14-17 min, 0% B, and 17-20 min, 0% B. MGO level is expressed as microgram per gram of extract.

### Results

TABLE 2
Levels of TPC, FRAP and MGO in the Leptospermum extract.

| | TPC (mgGAE/g) | FRAP (mgTE/g) | MGO (μg/g) |
|---|---|---|---|
| Leptospermum | 187.9 ± 40.7 | 320.7 ± 58.7 | 82.9 ± 8.1 |
| extract | | | |

Values are means ± standard deviations.
GAE: gallic acid equivalent,
TE Trolox equivalent

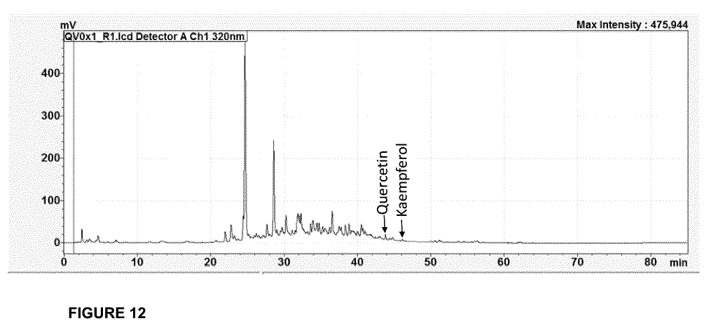

As shown in Table 2, the Leptospermum extract has a total phenolic content ranging from 132.9 to 255.5 mgGAE/g of the extract. The range of phenolic content is significantly higher than that of ginseng root extract (Malathy et al. (2021), Appl. Sci), as well as selected Mexican medicinal plant extracts (Wong-Paz et al. (2015), Asian Pacific Journal of Tropical Medicine). These studies reported TPC ranging from as high as 30-100 mgGAE/g but as low as 0-5 mgGAE/g. This suggests that the Leptospermum extracts contain a significantly higher amount of phenolic bioactive compounds than comparable medicinal plant extracts. The Leptospermum extract also exhibits strong antioxidant activity with Ferric Antioxidant Power ranging from 279.2 to 362.3 mgTE/g of the extract. In addition, scanning using a photo diode array (PDA) detector revealed that the Leptospermum extract has over 30 major individual compounds, which can exhibit antioxidant activity and have impact on cancer cells (as shown in FIG. 12). MGO was also detected in Leptospermum extract ranging from 74.9 to 100.8 μg/g. Other notable bioactive compounds present in the HPLC chromatogram of FIG. 12 were identified as quercetin and kaempferol.

### SUMMARY

The results indicate that Leptospermum extracts have utility in reducing fat mass and for body weight loss. Further, animals fed with Leptospermum extract exhibited decreased fasting blood glucose levels as compared to control animals. Lastly, liver toxicity and histological analysis revealed that administration of the Leptospermum extracts did not result in any adverse side effects, suggesting that the extracts were safe for consumption even when administered over the course of 28 days. In vitro assays revealed that cancer cells treated with the Leptospermum extracts underwent cell death, exhibited reduced cell migration, and displayed increased basal respiratory capacity (as a measure of energy expenditure) in cancer cells as compared to controls. Taken together, the results indicate that the Leptospermum extracts have a specific effect on cancer cells and supports its therapeutic utility in the treatment of cancer. Additionally, Leptospermum extracts were shown to have potent antioxidative properties.

The examples illustrate novel methods and uses for Leptospermum extracts in the treatment or prevention of cancer, metabolic syndromes including reducing body fat and overall weight, utility in reducing blood glucose levels in order to maintain glucose levels within healthy ranges and finally potent antioxidative properties that support its use in the treatment of diseases or conditions associated with oxidative stress.

The Leptospermum extract is shown in the Examples to be one that results in cell death of at least 50% in cancer cell lines remaining following a cytotoxic assay (cell proliferation). The percentage of cancer cells that have undergone cell death in some embodiments is at least 70%, or even at least 80%.

The Leptospermum extract is shown to be one that provides a reduction in tumour cell count in cancer subjects of at least 10% as compared to controls. The reduction in tumour cell count in some embodiments is at least 20%, or even at least 50%.

The Leptospermum extract is shown to be one that provides a reduction in weight of tumours in cancer subjects of at least 1% relative to body weight, as compared to controls. The reduction in weight of tumours in some embodiments is at least 2%, or even at least 3% relative to body weight.

The Leptospermum extract is shown to be one that provides an increase in remaining body weight in cancer subjects of at least 1% as compared to controls. The increase in remaining body weight in some embodiments is at least 2%, or even at least 5%.

The Leptospermum extract is shown to be one that provides a reduction in body weight of at least 1% as compared to controls. The reduction in body weight in some embodiments is at least 2%, or even at least 5%.

The Leptospermum extract is shown to be one that provides a reduction in fat mass of at least 1% as compared to controls. The reduction in fat mass in some embodiments is at least 2%, or even at least 5%.

The Leptospermum extract is shown to be one that provides a reduction in glucose levels of at least 1 mmol/L as compared to controls. The reduction in glucose levels in some embodiments is at least 2 mmol/L, or even at least 3 mmol/L.

The Leptospermum extract is shown to be one that reduces cell migration of cancer cells in a wound-healing (scratch) assay by at least 10% compared to controls. The reduction in cell migration of cancer cells in some embodiments is at least 20%, or even at least 50%.

The Leptospermum extract is shown to be one that provides a basal respiratory capacity of at least 400 pmol/min as compared to controls, based on the Seahorse XF Cell Mito Stress Test. The basal respiratory capacity in some embodiments is at least 500 pmol/min, or even at least 600 pmol/min.

The Leptospermum extract is shown to be one that has a total phenolic content of at least 100 mg/gram of gallic acid (mg of gallic acid equivalent per g dry weight), as determined using the Folin-Ciocalteu method. The total phenolic content in some embodiments is at least 125 mg/g gallic acid, or even at least 150 mg/g gallic acid.

The Leptospermum extract is shown to be one that provides a ferric-reducing antioxidant potential (FRAP) of at least 500,000 mg/ml. The FRAP may, in some embodiments, be at least 700,000 mg/ml, or even at least 1,000,000 mg/ml.

1.-42. (canceled)

43. A method of treating or preventing lung cancer in a subject in need thereof comprising administering a composition comprising a polar solvent-extract from a plant of the genus Leptospermum, wherein the plant is selected from the group consisting of Leptospermum liversidgei, Leptospermum polygahfolium, Leptospermum laevigatum, Leptospermum continentale, Leptospermum amboinense, Leptospermum arachnoides, Leptospermum brachyandrum, Leptospermum brevipes, Leptospermum coriaceum, Leptospermum deuense, Leptospermum epacridoideum, Leptospermum juniperinum, Leptospermum lanigerum, Leptospermum luehmannii, Leptospermum macrocarpum, Leptospermum minutifolium, Leptospermum multicaule, Leptospermum myrsinoides, Leptospermum nitidum, Leptospermum obovatum, Leptospermum oligandrum, Leptospermum parvifolium, Leptospermum petersonii, Leptospermum polygahfolium ‘Pacific Beauty’, Leptospermum purpurascens, Leptospermum rotundifolium, Leptospermum rotundifolium ‘Julie Ann’, Leptospermum sericeum, Leptospermum speciosum, Leptospermum spectabile, Leptospermum spinescens, Leptospermum squarrosum, Leptospermum trinervium, Leptospermum turbinatum, Leptospermum variabile, and Leptospermum wooroonooran, and wherein the polar solvent is water.

44. A method of treating mesothelioma in a subject in need thereof comprising administering a composition comprising a polar solvent-extract from a plant of the genus Leptospermum, wherein the plant is selected from the group consisting of Leptospermum scoparium, Leptospermum liversidgei, Leptospermum polygahfolium, Leptospermum laevigatum, Leptospermum continentale, Leptospermum amboinense, Leptospermum arachnoides, Leptospermum brachyandrum, Leptospermum brevipes, Leptospermum coriaceum, Leptospermum deuense, Leptospermum epacridoideum, Leptospermum juniperinum, Leptospermum lanigerum, Leptospermum luehmannii, Leptospermum macrocarpum, Leptospermum minutifolium, Leptospermum multicaule, Leptospermum myrsinoides, Leptospermum nitidum, Leptospermum obovatum, Leptospermum oligandrum, Leptospermum parvifolium, Leptospermum petersonii, Leptospermum polygalifolium ‘Pacific Beauty’, Leptospermum purpurascens, Leptospermum rotundifolium, Leptospermum rotundifolium ‘Julie Ann’, Leptospermum sericeum, Leptospermum speciosum, Leptospermum spectabile, Leptospermum spinescens, Leptospermum squarrosum, Leptospermum trinervium, Leptospermum turbinatum, Leptospermum variabile, and Leptospermum wooroonooran, wherein the polar solvent extract is an aqueous extract.

45. The method of claim 44, wherein the mesothelioma is a mesothelioma of the lung, abdomen or heart.

46. The method of claim 43, wherein the extract comprises any one or more actives selected from the group consisting of: aromadendrin glucoside, kaempferol rhamnoside, quercetin rhamnoside, vindoline, 5-dihydroxy-6-methyl-7-methoxyflavanone, and methylglyoxal.

47. The method of claim 43, wherein the composition is in a form for oral administration.

48. The method of claim 43, wherein the extract is in powder form.

49. The method of claim 43, wherein the extract is in encapsulated form.

50. The method of claim 43, further comprising administering an additional therapeutic agent selected from the group consisting of: a chemotherapeutic agent, an anti-inflammatory agent, an immunomodulatory agent, a neurotropic factor, an agent for treating a cardiovascular disease, an agent for treating liver disease, an anti-viral agent, an agent for treating hyperglycemia, and an agent for treating immunodeficiency disorders.

51. The method of claim 43, wherein the composition is a food composition.

52. The method of claim 51, wherein the food composition comprises a honey product.

53. The method of claim 52, wherein the honey product is from Leptospermum scoparum.

54. The method of claim 52, wherein the honey product is present within the range of about 10 to 99 wt %, based on the total weight of the composition.

55. The method of claim 43, wherein the composition further comprises one or more of: suspending agents, emulsifying agents, gelling agents, wetting agents, fillers, binders, preservatives, flavouring, humectant, colouring and/or sweetening agents.

56. The method of claim 43, comprising administering the extract once daily.

57. The method of claim 43, comprising administering the extract at least once daily for a period of 1 month.

58. The method of claim 44, wherein the extract comprises any one or more actives selected from the group consisting of: aromadendrin glucoside, kaempferol rhamnoside, quercetin rhamnoside, vindoline, 5-dihydroxy-6-methyl-7-methoxyflavanone, and methylglyoxal.

59. The method of claim 44, wherein the composition is in a form for oral administration.

60. The method of claim 44, wherein the extract is in powder form.

61. The method of claim 44, wherein the extract is in encapsulated form.

62. The method of claim 44, further comprising administering an additional therapeutic agent selected from the group consisting of: a chemotherapeutic agent, an anti-inflammatory agent, an immunomodulatory agent, a neurotropic factor, an agent for treating a cardiovascular disease, an agent for treating liver disease, an anti-viral agent, an agent for treating hyperglycemia, and an agent for treating immunodeficiency disorders.

63. The method of claim 44, wherein the composition is a food composition.

64. The method of claim 63, wherein the food composition comprises a honey product.

65. The method of claim 64, wherein the honey product is from Leptospermum scoparum.

66. The method of claim 64, wherein the honey product is present within the range of about 10 to 99 wt %, based on the total weight of the composition.

67. The method of claim 44, wherein the composition further comprises one or more of: suspending agents, emulsifying agents, gelling agents, wetting agents, fillers, binders, preservatives, flavouring, humectant, colouring and/or sweetening agents.

68. The method of claim 44, comprising administering the extract once daily.

69. The method of claim 44, comprising administering the extract at least once daily for a period of 1 month.
